SEQUENCE LISTING (7 sequences)

SEQ ID NO: 1 - protein, 404 aa, Aspergillus oryzae
MVILSKVAAVAVGLSTVASALPTGPSHSPHARRGFTINQITRQTARVGPKTASFPAIYSRALAKYGGTVPAHLKSAVASGHGTVVTSPEPNDIEYLTPVNIGGTTLNLDFDTGSADLWVFSEELPKSEQTGHDVYKPSGNASKIAGASWDISYGDGSSASGDVYQDTVTVGGVTAQGQAVEAASKISDQFVQDKNNDGLLGLAFSSINTVKPKPQTTFFDTVKDQLDAPLFAVTLKYHAPGSYDFGFIDKSKFTGELAYADVDDSQGFWQFTADGYSVGKGDAQKAPITGIADTGTTLVMLDDEIVDAYYKQVQGAKNDASAGGYVFPCETELPEFTVVIGSYNAVIPGKHINYAPLQEGSSTCVGGIQSNSGLGLSILGDVFLKSQYVVFDSQGPRLGFAAQA

SEQ ID NO: 2 - protein, 394 aa, Aspergillus niger
MVVFSKTAALVLGLSSAVSAAPAPTRKGFTINQIARPANKTRTINLPGMYARSLAKFGGTVPQSVKEAASKGSAVTTPQNNDEEYLTPVTVGKSTLHLDFDTGSADLWVFSDELPSSEQTGHDLYTPSSSATKLSGYTWDISYGDGSSASGDVYRDTVTVGGVTTNKQAVEAASKISSEFVQNTANDGLLGLAFSSINTVQPKAQTTFFDTVKSQLDSPLFAVQLKHDAPGVYDFGYIDDSKYTGSITYTDADSSQGYWGFSTDGYSIGDGSSSSSGFSAIADTGTTLILLDDEIVSAYYEQVSGAQESEEAGGYVFSCSTNPPDFTVVIGDYKAVVPGKYINYAPISTGSSTCFGGIQSNSGLGLSILGDVFLKSQYVVFNSEGPKLGFAAQA

SEQ ID NO: 3 - protein, 378 aa, Aspergillus melleus
MRFPIYATALAATASALVIGERASSEDRYILELAPGVTKVVTEAEKWSLKAEGKRFFDITDEVSTIKTANVEDSQQQLAVTYPSKVQYKDTVEKLIPQLSKANFQTVLKPFSGFHNRYYKSNYGKQSSEWLQAQIQSVVDASGAKGITVKPFSHSWTQSSIIATIPGKSNKIVVLGAHQDSINQASPSTGRAPGADDDGSGVVTILETLRVLLKDPKVAAGEATNTIEFHFYSAEEGGLLGSQAIFRQYSSQSKQVVAMLQQDMTGYTEGTKKAGKPEAIGVLTDYVDAGLTKFLKTIIDTYTTISWVESQCGYACSDHASANRYGYPSSFAFESEFGDDSPYIHTAQDTISTVDFDHVLQHARLSLGFAYELGFASL

SEQ ID NO: 4 - protein, 499 aa, Aspergillus oryzae
MMVAWWSLFLYGLQVAAPALAATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 5 - protein, 549 aa, Candida cylindraceae
MELALALSLIASVAAAPTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYRVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

SEQ ID NO: 6 - protein, 640 aa, Aspergillus niger
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 7 - protein, 404 aa, Aspergillus melleus
MPSFKQTLLLLGAMLPAVFGAPVENTRRASEKIAGKYIVTFKSGIDTAKIEQHTTWATNIHKRNLARRDASDDSDLPVGIEKNFKIKNFAAYFGSFDDSTIEEIRKSADVAHIEEDQVWYLDALTTQTGATWGLGSISHKGESSTSYVYDSSAGEGTYGYVVDTGINVDHSEFGGRASLAYNAVGGQHVDSVGHGTHVAGTIGGKTYGVSKKANLLSVKVFQGESSSTSIILDGYNWAANDIVSKSRTGKAAINLSLGGGYSYAFNQAVENAFDEGVLTVVAAGNENSDAGDTSPASAPNALTVAASTNRNARASFSNYGSVVDVFAPGQDIKSAWIGSSSATNTISGTSMATPHIVGLAIYLQALEGLTSPAAVTKRIKELATSGVVTDVKGSPNLLAYNGAA

(19) United States
(12) Patent Application Publication
(10) Pub. No.: US 2024/0382569 A1
(43) Pub. Date: Nov. 21, 2024

(54) FUNGAL ENZYME MIXTURES AND USES THEREOF

(71) Applicant: Bio-Cat, Inc., Troy, VA (US)

(72) Inventors: Sean Michael GARVEY, Zion Crossroads, VA (US); Kelly Marie TINKER, Columbia, VA (US); Morgan Dabney HOLLINS, Bumpass, VA (US); Justin Lamont GUICE, Midlothian, VA (US); Christopher SCHULER, Charlottesville, VA (US)

(21) Appl. No.: 18/688,983

(22) PCT Filed: Sep. 2, 2022
(86) PCT No.: PCT/US2022/042501

§ 371 Date: Mar. 4, 2024

Related U.S. Application Data
(60) Provisional application No. 63/240,255, filed on Sep. 2, 2021.

(51) Int. Cl. A61K 38/48; A61K 38/46; A61K 38/47; A61P 1/14

(52) U.S. Cl. CPC A61K 38/488; A61K 38/465; A61K 38/47; A61K 38/4813; A61K 38/482; A61P 1/14; C12Y 301/01003; C12Y 302/01001; C12Y 302/01003; C12Y 304/21063; C12Y 304/23018

(57) ABSTRACT

Novel fungal enzyme compositions, and more particularly, enzyme mixtures comprising a plurality of fungal enzymes (e.g., from Aspergillus and Candida) are provided. The disclosure further relates to dietary supplements, foods and beverages containing these enzyme mixtures, and methods of making and using the same.

### CROSS-REFERENCE TO RELATED APPLICATIONS

This application claims priority to U.S. Provisional Application No. 63/240,255, filed on Sep. 2, 2021, the disclosure of which is hereby incorporated by reference in its entirety.

### TECHNICAL FIELD

Novel fungal enzyme compositions, and more particularly, enzyme mixtures comprising a plurality of fungal enzymes (e.g., from Aspergillus and Candida) are provided. The disclosure further relates to dietary supplements and foods containing these enzyme mixtures, methods of making and using the same.

### SUMMARY OF VARIOUS ASPECTS

In a general aspect, the present disclosure relates to combinations of enzymes obtained from members of the genus Aspergillus, such as A. niger, A. oryzae, or A. melleus, and the genus Candida (or Rhizopus), which are capable of digesting dietary proteins, fats, and carbohydrates from various sources. In some exemplary aspects, the enzyme mixtures described herein may be used as ingredients in dietary supplements or foods to promote protein digestion, to increase postprandial plasma amino acid concentrations, to promote lipid digestion, to increase postprandial plasma fatty acid concentrations, to promote carbohydrate digestion, to reduce the maximum concentration of postprandial plasma blood glucose, to flatten the postprandial plasma glucose concentration area-under-the-curve, to increase postprandial serum iron concentrations, or any combination thereof, following consumption of a meal, food, beverage, or dietary supplement. In some exemplary aspects, the enzyme mixture described herein may be used in dietary supplements, foods or beverages to promote digestive health and lessen the frequency, duration, or severity of gastrointestinal (GI) symptoms including burping, cramping, pain, distension, bloating, flatulence, gas, nausea, reflux, heartburn, borborygmus (i.e., stomach, rumblings), and diarrhea. In some exemplary aspects, the enzyme mixture described herein may be used in dietary supplements, foods or beverages to improve bowel function according to improvements in any of stool frequency, stool consistency, and ease of stool passage. In some exemplary aspects, the enzyme mixture described herein may be used in dietary supplements, foods or beverages to improve sleep quality. Moreover, the mixtures of enzymes described herein are stable and maintain activity over a broad range of temperatures and pH levels, providing additional options for commercial and industrial applications.

In other general aspects, the disclosure provides methods of administering a dietary supplement comprising the disclosed enzyme mixtures to increase plasma concentrations of essential amino acids (EAA), branched chain amino acids (BCAA), fatty acids, and/or iron absorption, and/or increase or modify plasma concentrations of glucose, following consumption of a meal, food, beverage, or dietary supplement.

Methods of using the disclosed dietary supplements containing the enzyme mixture are also provided, including methods of increasing or maintaining muscle mass, decreasing muscle breakdown, improving GI tolerance, decreasing GI symptoms, improving bowel function, improving sleep quality, or combinations thereof, by administering a dietary supplement containing the enzyme mixture described herein, alone or as part of a dietary supplement or food.

Additional aspects will be readily apparent to one of skill in light of the totality of the disclosure.

### BRIEF DESCRIPTION OF THE DRAWINGS

FIG. 1A is a graph illustrating the relative activity of Fungal Protease A across various pH levels, and FIG. 1B is a graph illustrating the relative activity of Fungal Protease A across various temperature levels.

FIG. 2A is a graph illustrating the relative activity of Fungal Acid Protease across various pH levels, and FIG. 2B is a graph illustrating the relative activity of Fungal Acid Protease across various temperature levels.

FIG. 3A is a graph illustrating the relative activity of Protease AM across various pH levels, and FIG. 3B is a graph illustrating the relative activity of Protease AM across various temperature levels.

FIG. 4A is a graph illustrating the relative activity of Yeast Lipase across various pH levels, and FIG. 4B is a graph illustrating the relative activity of Yeast Lipase across various temperature levels.

FIG. 5A is a graph illustrating the relative activity of Fungal Amylase across various pH levels, and FIG. 5B is a graph illustrating the relative activity of Fungal Amylase across various temperature levels.

FIG. 6A is a graph illustrating the relative activity of Glucoamylase across various pH levels, and FIG. 6B is a graph illustrating the relative activity of Glucoamylase across various temperature levels.

FIG. 7A is a graph illustrating the relative activity of Rhizopus lipase across various pH levels, and FIG. 7B is a graph illustrating the relative activity of Rhizopus lipase across various temperature levels.

FIGS. 8A and 8B are bar charts showing the concentrations of free amino nitrogen (FAN) following simulated salivary-gastric (SG) digestion (FIG. 8A), and full salivary-gastric-intestinal (SGI) digestion (FIG. 8B) of the canned test meal (CTM), oral nutritional supplement (ONS), and pea protein isolate (PPI) substrates by BC-006, compared to control with substrate and only the endogenous porcine enzymes, as described in Example 1 (**, P<0.01; ***, P<0.001; ****, P<0.0001).

FIGS. 9A-9C are bar charts showing leucine release, branched chain amino acid (BCAA) release, and essential amino acid (EAA) release, following simulated salivary-gastric (SG) digestion of the canned test meal (FIG. 9A), oral nutritional supplement (FIG. 9B), and pea protein isolate (FIG. 9C) substrates by BC-006, compared to control with substrate and only the endogenous porcine enzymes, as described in Example 1 (**, P<0.01; ***, P<0.001; ****, P <0.0001).

FIGS. 10A and 10B are bar charts showing glycerol release following simulated salivary-gastric (SG) digestion (FIG. 10A), and full salivary-gastric-intestinal (SGI) digestion (FIG. 10B) of the canned test meal (CTM) and oral nutritional supplement (ONS) substrates by BC-006, compared to control with substrate and only the endogenous porcine enzymes, as described in Example 2 (**, P<0.01; ***, P<0.001).

FIG. 11 is a bar chart showing the concentration of total triglycerides following full salivary-gastric-intestinal (SGI) digestion of butter by BC-006, compared to control with butter and only the endogenous porcine enzymes, as described in Example 2 (*, P<0.05).

FIGS. 12A and 12B are bar charts showing maltose release following simulated salivary-gastric (SG) digestion (FIG. 12A), and full salivary-gastric-intestinal (SGI) digestion (FIG. 12B) of the canned test meal (CTM) and oral nutritional supplement (ONS) substrates by BC-006, compared to control with same substrate and only the endogenous porcine enzymes, as described in Example 3 (***, P<0.001; ****, P<0.0001).

FIGS. 13A and 13B are bar charts showing glucose release following simulated salivary-gastric (SG) digestion (FIG. 13A), and full salivary-gastric-intestinal (SGI) digestion (FIG. 13B) of the canned test meal (CTM), oral nutritional supplement (ONS), and pea protein isolate (PPI) substrates by BC-006, compared to control with substrate and only the endogenous porcine enzymes, as described in Example 3 (**, P<0.01; ***, P<0.001; ****, P<0.0001).

### DETAILED DESCRIPTION OF VARIOUS ASPECTS

The present disclosure relates to enzyme mixtures comprising a plurality of fungal enzymes obtained from members of the genus Aspergillus (e.g., from A. oryzae, A. niger, and A. melleus) and the species Candida cylindracea (or Rhizopus oryzae). These enzyme mixtures may be administered to a subject as a dietary supplement (e.g., to improve protein digestion or the absorption of amino acids, EAAs, and/or BCAAs, to improve fat digestion or the absorption of fatty acids, to improve carbohydrate digestion or the absorption of glucose, to improve postprandial iron levels, to improve GI tolerance, to decrease GI symptoms, to improve bowel function, and/or to improve sleep quality).

Proteins are high molecular weight polymers composed of multiple amino acids linked by peptide bonds. These bonds must be cleaved in order for protein to be absorbed and utilized by a human or other organism, with such cleavage typically being performed by endogenous proteolytic enzymes of the GI tract that separate the polypeptides into its constituent free amino acids. Amino acids may be classified as essential or non-essential for any given organism, depending on whether an organism is capable of synthesizing the given amino acid. For a dietary regimen to be considered adequate for the support of normal physiological functions, it should contain all EAAs in the appropriate levels and in proper proportions. For humans, the nine EAAs are leucine, isoleucine, valine, methionine, tryptophan, phenylalanine, threonine, lysine and histidine.

Three of the EAAs (valine, leucine and isoleucine) have aliphatic side chains with a branch (i.e., a central carbon atom bound to three or more carbon atoms). These BCAAs are particularly notable because these amino acids are an important nutritional factor for proper muscle physiology and muscle protein synthesis (MPS). See Kimball & Jefferson, “Signaling Pathways and Molecular Mechanisms through which Branched-Chain Amino Acids Mediate Translational Control of Protein Synthesis.” The Journal of Nutrition. 2006. 136(1 Suppl), 227S-2231S. Reports further indicate that athletic and exercise performance may be improved by oral BCAA supplementation. See e.g., Glynn et al., “Excess Leucine Intake Enhances Muscle Anabolic Signaling but Not Net Protein Anabolism in Young Men and Women.” The Journal of Nutrition. 2010. 140(11), 1970-1976; Sharp et al., “Amino Acid Supplements and Recovery from High-Intensity Resistance Training.” Journal of Strength and Conditioning Research. 2010. 24(4), 1125-1130. In older adults, who are at risk of severe muscle wasting (i.e., sarcopenia), protein or BCAA supplementation in combination with exercise has also been shown to support performance, muscle mass, and strength. See e.g., Tieland et al., “Protein Supplementation Increases Muscle Mass Gain During Prolonged Resistance-Type Exercise Training in Frail Elderly People: A Randomized, Double-Blind, Placebo-Controlled Trial.” Journal of the American Medical Directors Association. 2012. 3(8), 713-719; Zdzieblik et al., “Collagen Peptide Supplementation in Combination with Resistance Training Improves Body Composition and Increases Muscle Strength in Elderly Sarcopenic Men: A Randomised Controlled Trial.” The British Journal of Nutrition. 2015, 114(8), 1237-1245; and Ikeda, et al., “Effects and Feasibility of Exercise Therapy Combined with Branched-Chain Amino Acid Supplementation on Muscle Strengthening in Frail and Pre-Frail Elderly People Requiring Long-Term Care: A Crossover Trial.” Applied Physiology, Nutrition, and Metabolism. 2016. 41(4), 438-445.

In view of the above, there is a commercial interest in dietary supplements and food additives that contain EAAs and BCAAs (e.g., protein powders and energy drinks directed to athletes and/or older adults at risk for protein malnutrition). These supplements minimally include a raw protein source (e.g., whey protein concentrate, soy protein isolate, pea protein isolate), and the raw protein source ingredient may be further processed by using a proteolytic enzyme or combination of enzymes (e.g., to manufacture a whey protein hydrolysate ingredient). See Nasri, “Protein Hydrolysates and Biopeptides: Production, Biological Activities, and Applications in Foods and Health Benefits. A Review.” Advances in Food and Nutrition Research. 2017. 81, 109-159. A supplement product with minimally processed protein or protein hydrolysate may further be modified by the inclusion of leucine, other BCAAs, or other EAAs obtained from a second process or source. The manufacturing of such products is therefore complicated by the fact that amino acids must typically be obtained from multiple sources and mixed together to obtain a product which has the desired profile and ratios (e.g., enriched in BCAAs).

The present disclosure provides enzyme mixtures that simplify this process by administering the enzymes alongside meals, whole foods, oral nutritional supplements (ONS), and/or protein supplements to improve amino acid release in the GI tract. Use of these enzyme mixtures reduces the complexity and manufacturing costs associated with having to manufacture protein hydrolysates and/or obtain amino acids from different sources.

### Enzyme Mixtures Comprising a Plurality of Fungal Enzymes

In one general aspect, the present disclosure provides an enzyme mixture comprising a plurality of fungal enzymes obtained from members of the genus Aspergillus (e.g., a combination of at least three proteases obtained from A. oryzae, A. niger, or A. melleus) and the species Candida cylindracea. One or more of these enzymes may possess exopeptidase, endopeptidase, lipase activity, alpha-amylase activity, and/or glucoamylase activity alone or operating in combination with other enzymes in the mixture. In some exemplary aspects, the mixture has enzymatic activity across a pH range spanning from 2.0 to 9.0, or any range of integer values therein. In other aspects, the relative activity of the mixture may be >30% across a temperature range of 20 to 70° C., or >50% across a temperature range of 40 to 65° C. In other aspects, the mixture is stable over a temperature range of approximately 20 to 70° C.

In other aspects, the enzyme mixture is capable of digesting a food or protein source (e.g., grilled chicken, plant protein) and increasing the release of amino acids, EAAs, BCAAs, fatty acids, and/or glucose in the stomach and/or intestine. As such, the enzyme mixtures described herein may be used to improve the absorption of amino acids (e.g., BCAAs, EAAs, leucine), fatty acids, and/or glucose by a human or that consumes a dietary supplement or food comprising the enzyme mixture, by increasing the amounts of free amino acids, and BCAAs and EAAs in particular, fatty acids, and/or glucose released during digestion.

As used herein, references to hemoglobin unit tyrosine base units (HUT), stable acid protease units (SAPU), leucine aminopeptidase units (LAPU), Fédération Internationale Pharmaceutique units (FIP), and amyloglucosidase units (AGU), of the enzymes and enzyme mixtures described herein are calculated using the standard protocols described in the Food Chemicals Codex (FCC), 12^{th} Ed. (published Mar. 1, 2020), the contents of which is hereby incorporated by reference in its entirety. The Sandstedt, Kneen, and Blish method unit (SKB) of the enzymes and enzyme mixtures described herein are calculated using the SKB assay is described in Sandstedt et al., Cereal Chemistry. 1939. 16, 712-723, which is incorporated by reference in its entirety.

In some aspects, the enzyme mixture may comprise a mixture of six fungal enzymes (also referred to as “BC-006” in some of the charts and figures herein): BIO-CAT Fungal Protease A (a neutral protease obtained from A. oryzae with a predicted molecular weight of approximately 42 kilodalton (kDa), Chemical Abstracts Service (CAS) registration No. 9025-49-4, IUBMB Enzyme Commission (EC) No. 3.4.23.18), present at 240,000 HUT units per gram; BIO-CAT Acid Stable Protease A (an acid protease obtained from A. niger with a predicted molecular weight of approximately 41 kDa, CAS No. 9025-49-4, EC No. 3.4.23.18), present at 1,200 SAPU unit per gram; BIO-CAT Protease AM (including an alkaline protease obtained from A. melleus with a predicted molecular weight of approximately 42 kDa; CAS No. 9074-07-1; EC No. 3.4.21.63, and/or a leucine aminopeptidase obtained from A. melleus with a predicted molecular weight of approximately 41 kDa; EC No. 3.4.11.-), present at 200 LAPU units per gram; BIO-CAT Yeast Lipase (a lipase obtained from C. cylindracea with a predicted molecular weight of approximately 59 kDa, CAS No. 9001-62-1, EC No. 3.1.1.3), present at 12,000 FIP unit per gram; BIO-CAT Fungal Amylase (an alpha-amylase obtained from A. oryzae with a predicted molecular weight of approximately 55 kDa, CAS No. 9000-90-2, EC No. 3.2.1.1), present at 40,000 SKB per gram; and BIO-CAT Glucoamylase (or BIO-CAT amyloglucosidase, a glucoamylase obtained from A. niger with a predicted molecular weight of approximately 68 kDa, CAS No. 9032-08-0, EC No. 3.2.1.3), present at 100 AGU units per gram.

As described herein, this combination has been shown to release free amino nitrogen (FAN, a proxy for free amino acids), release glycerol (a proxy for triglyceride hydrolysis), decrease total triglycerides, release maltose, and release glucose in GI digestion simulations that model the wide range of pH levels that occur throughout the GI tract.

In other aspects, the lipase is from Rhizopus oryzae. In particular aspects, the Rhizopus lipase has a predicted molecular weight of approximately 42 kDa (CAS No. 9001-62-1, EC No. 3.1.1.3). Thus, in a particular aspect, the enzyme mixture comprises BIO-CAT Fungal Protease A (a neutral protease obtained from A. oryzae with a predicted molecular weight of approximately 42 kDa, CAS No. 9025-49-4, EC No. 3.4.23.18), BIO-CAT Acid Stable Protease A (an acid protease obtained from A. niger with a predicted molecular weight of approximately 41 kDa, CAS No. 9025-49-4, EC No. 3.4.23.18), BIO-CAT Protease AM (an alkaline protease obtained from A. melleus with a predicted molecular weight of approximately 42 kDa, CAS No. 9074 Jul. 1, EC No. 3.4.21.63, and/or a leucine aminopeptidase obtained from A. melleus with a predicted molecular weight of approximately 41 kDa, EC No. 3.4.11.-), BIO-CAT Lipase (a lipase obtained from R. oryzae with a predicted molecular weight of approximately 42 kDa, CAS No. 9001-62-1, EC No. 3.1.1.3), BIO-CAT Fungal Amylase (an alpha-amylase obtained from A. oryzae with a predicted molecular weight of approximately 55 kDa, CAS No. 9000-90-2, EC No. 3.2.1.1), and BIO-CAT Glucoamylase (or BIO-CAT Amyloglucosidase, a glucoamylase obtained from A. niger with a predicted molecular weight of approximately 68 kDa, CAS No. 9032-08-0, EC No. 3.2.1.3).

In other aspects, the Fungal Protease A comprises SEQ ID NO: 1, the Acid Stable Protease A comprises SEQ ID NO: 2, the Protease AM comprises SEQ ID NO: 3 and/or SEQ ID NO: 7, the Fungal Amylase comprises SEQ ID NO: 4, and the Yeast Lipase comprises SEQ ID NO: 5, and the Glucoamylase comprises SEQ ID NO: 6. See Description of Sequences.

In other aspects, the enzyme mixture comprises a plurality of fungal enzymes from the genus Aspergillus and the genus Candida, wherein the mixture comprises SEQ ID NOS: 1, 2, 3, 4, 5, 6, and 7. In other aspects, the enzyme mixture comprises SEQ ID NOS: 1, 2, 3, 4, 5, and 6. In other aspects, the enzyme mixture comprises SEQ ID NOS: 1, 2, 4, 5, 6, and 7.

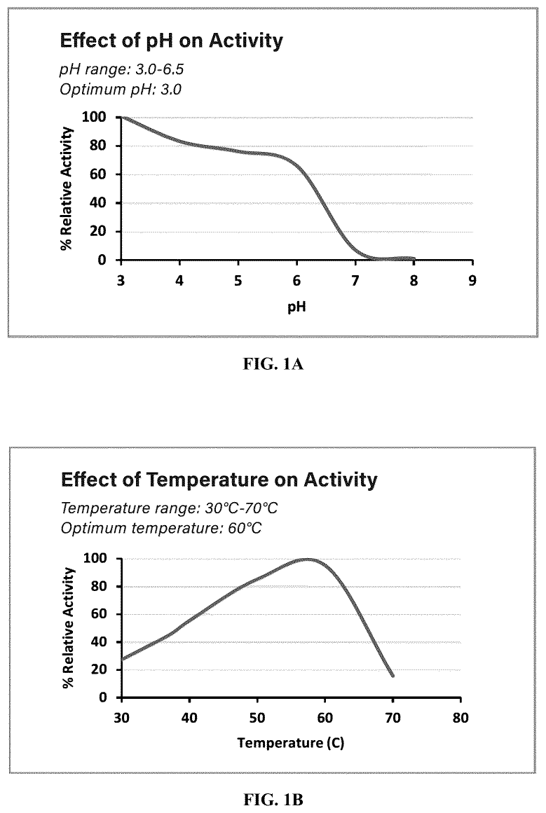

In other aspects, the enzyme mixture comprises a plurality of fungal enzymes from the genus Aspergillus and the genus Candida, wherein the mixture comprises a protease from A. oryzae, a protease from A. niger, a protease from A. melleus, an alpha-amylase from A. oryzae, a lipase from C. cylindracea, and a glucoamylase from A. niger. In other aspects, the enzyme mixture comprises a plurality of fungal enzymes, wherein the enzyme mixture comprises enzymes having the relative activities described in FIGS. 1-6 or 1-3 and 5-7.

In other aspects, the enzyme mixture comprises a plurality of fungal enzymes from the genus Aspergillus and the genus Candida, wherein the mixture comprises SEQ ID NO: 1, a protease obtained from A. niger, a protease obtained from A. melleus, SEQ ID NO: 4, SEQ ID NO: 5, and SEQ ID NO: 6. In another aspect, the enzyme mixture comprises SEQ ID NO: 1, a protease obtained from A. niger, SEQ ID NO: 3, SEQ ID NO: 4, SEQ ID NO: 5, and SEQ ID NO: 6. In another aspect, the enzyme mixture comprises SEQ ID NO: 1, a protease obtained from A. niger, SEQ ID NO: 4, SEQ ID NO: 5, SEQ ID NO: 6, and SEQ ID NO: 7. In another aspect, the enzyme mixture comprises SEQ ID NO: 1, SEQ ID NO: 2, a protease obtained from A. melleus, SEQ ID NO: 4, SEQ ID NO: 5, and SEQ ID NO: 6.

In other aspects, the enzyme mixture comprises a plurality of fungal enzymes from the genus Aspergillus and the genus Candida, wherein the mixture comprises SEQ ID NO: 1, a protease obtained from A. niger, a protease obtained from A. melleus, SEQ ID NO: 4, SEQ ID NO: 5, and a glucoamylase from A. niger. In another aspect, the enzyme mixture comprises SEQ ID NO: 1, a protease obtained from A. niger, SEQ ID NO: 3, SEQ ID NO: 4, SEQ ID NO: 5, and a glucoamylase from A. niger. In another aspect, the enzyme mixture comprises SEQ ID NO: 1, a protease obtained from A. niger, SEQ ID NO: 4, SEQ ID NO: 5, a glucoamylase from A. niger, and SEQ ID NO: 7. In another aspect, the enzyme mixture comprises SEQ ID NO: 1, SEQ ID NO: 2, a protease obtained from A. melleus, SEQ ID NO: 4, SEQ ID NO: 5, and a glucoamylase from A. niger.

In other aspects, the enzyme mixture comprises a plurality of fungal enzymes from the genus Aspergillus and the genus Candida, wherein the mixture comprises a protease obtained from A. oryzae, a protease obtained from A. niger, a protease from A. melleus, SEQ ID NO: 4, SEQ ID NO: 5, and SEQ ID NO: 6. In another aspect, the enzyme mixture comprises a protease obtained from A. oryzae, a protease obtained from A. niger, SEQ ID NO: 3, SEQ ID NO: 4, SEQ ID NO: 5, and SEQ ID NO: 6. In another aspect, the enzyme mixture comprises a protease obtained from A. oryzae, a protease obtained from A. niger, SEQ ID NO: 4, SEQ ID NO: 5, SEQ ID NO: 6, and SEQ ID NO: 7. In another aspect, the enzyme mixture comprises a protease obtained from A. oryzae, SEQ ID NO: 2, SEQ ID NO: 3, SEQ ID NO: 4, SEQ ID NO: 5, SEQ ID NO: 6. In another aspect, the enzyme mixture comprises a protease obtained from A. oryzae, SEQ ID NO: 2, SEQ ID NO: 4, SEQ ID NO: 5, SEQ ID NO: 6, and SEQ ID NO: 7. In another aspect, the enzyme mixture comprises a protease obtained from A. oryzae, SEQ ID NO: 2, a protease obtained from A. melleus, SEQ ID NO: 4, SEQ ID NO: 5, and SEQ ID NO: 6.

In other aspects, the enzyme mixture comprises a plurality of fungal enzymes from the genus Aspergillus and the genus Candida, wherein the mixture comprises a protease obtained from A. oryzae, a protease obtained from A. niger, a protease from A. melleus, SEQ ID NO: 4, SEQ ID NO: 5, and a glucoamylase from A. niger. In another aspect, the enzyme mixture comprises a protease obtained from A. oryzae, a protease obtained from A. niger, SEQ ID NO: 3, SEQ ID NO: 4, SEQ ID NO: 5, and a glucoamylase from A. niger. In another aspect, the enzyme mixture comprises a protease obtained from A. oryzae, a protease obtained from A. niger, SEQ ID NO: 4, SEQ ID NO: 5, a glucoamylase from A. niger, and SEQ ID NO: 7. In another aspect, the enzyme mixture comprises a protease obtained from A. oryzae, SEQ ID NO: 2, SEQ ID NO: 3, SEQ ID NO: 4, SEQ ID NO: 5, a glucoamylase from A. niger. In another aspect, the enzyme mixture comprises a protease obtained from A. oryzae, SEQ ID NO: 2, SEQ ID NO: 4, SEQ ID NO: 5, a glucoamylase from A. niger, and SEQ ID NO: 7. In another aspect, the enzyme mixture comprises a protease obtained from A. oryzae, SEQ ID NO: 2, a protease obtained from A. melleus, SEQ ID NO: 4, SEQ ID NO: 5, and a glucoamylase from A. niger.

In some aspects, the enzyme mixture comprises a plurality of fungal enzymes from the genus Aspergillus and the genus Candida, wherein the mixture comprises SEQ ID NO: 1, SEQ ID NO: 2, SEQ ID NO: 3, SEQ ID NO: 4, SEQ ID NO: 5, SEQ ID NO: 6, and/or SEQ ID NO: 7, or a fragment or variant of any of these enzymes. For example, in some aspects, the proteolytic enzyme mixture comprises at least one variant enzyme which shares at least 90, 91, 92, 93, 94, 95, 96, 97, 98 or 99% full-length sequence identity with any one of SEQ ID NOs: 1-7, and which retains one or more of the enzymatic activities of SEQ ID NOs: 1-7. For example, a polypeptide sequence may differ from any one of SEQ ID NOs: 1-7 by the presence of one or more conservative or non-conservative substitutions which do not impact the catalytic or other activity of the enzyme. As used herein, the term “sequence identity” refers to the degree to which two polypeptide sequences are identical (i.e., on a residue-by-residue basis) over the window of comparison. The percentage of sequence identity is calculated by comparing two optimally aligned sequences over the window of comparison, determining the number of positions at which identical residues occurs in both sequences to yield the number of matched positions, dividing the number of matched positions by the total number of positions in the window of comparison (i.e., the window size), and multiplying the result by 100 to yield the percentage of sequence identity.

In some aspects, the enzyme mixture comprises a plurality of fungal enzymes from the genus Aspergillus and the genus Candida, wherein the mixture comprises at least one enzyme which shares at least 90, 91, 92, 93, 94, 95, 96, 97, 98 or 99% full-length sequence identity with a portion of the sequence of SEQ ID NOs: 1-7. In some aspects, the enzyme mixture comprises at least two, three, four, five, six, or seven enzymes which share at least 90, 91, 92, 93, 94, 95, 96, 97, 98 or 99% full-length sequence identity with a portion of the sequence of SEQ ID NOs: 1-7. In some aspects, the enzyme mixture comprises at least one, two, three, four, five, or six enzymes which share at least 90, 91, 92, 93, 94, 95, 96, 97, 98 or 99% full-length sequence identity with a portion of the sequence of SEQ ID NOs: 1-6. In some aspects, the enzyme mixture comprises at least one, two, three, four, five, or six enzymes which share at least 90, 91, 92, 93, 94, 95, 96, 97, 98 or 99% full-length sequence identity with a portion of the sequence of SEQ ID NOs: 1, 2, and 4-7.

In other aspects, the enzymes are derived from fungal sources that have been genetically modified (e.g., a foreign gene has been introduced into the fungal source organism to improve production, or the gene encoding the fungal enzyme has been introduced into a common microbial recombinant production organism, e.g., Bacillus subtilis, Saccharomyces cerevisiae, Escherichia coli, Pichia pastoris, Yarrowia lipolytica). In other aspects, the enzymes are derived from fungal sources that have not been genetically modified.

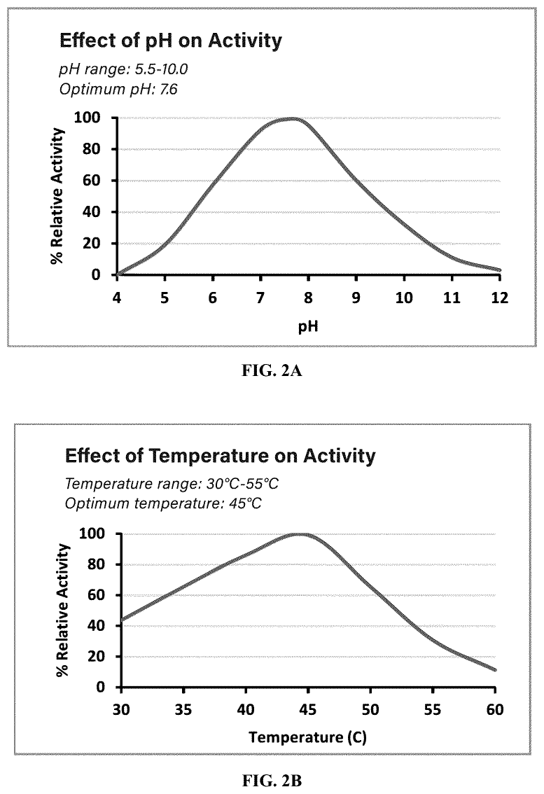

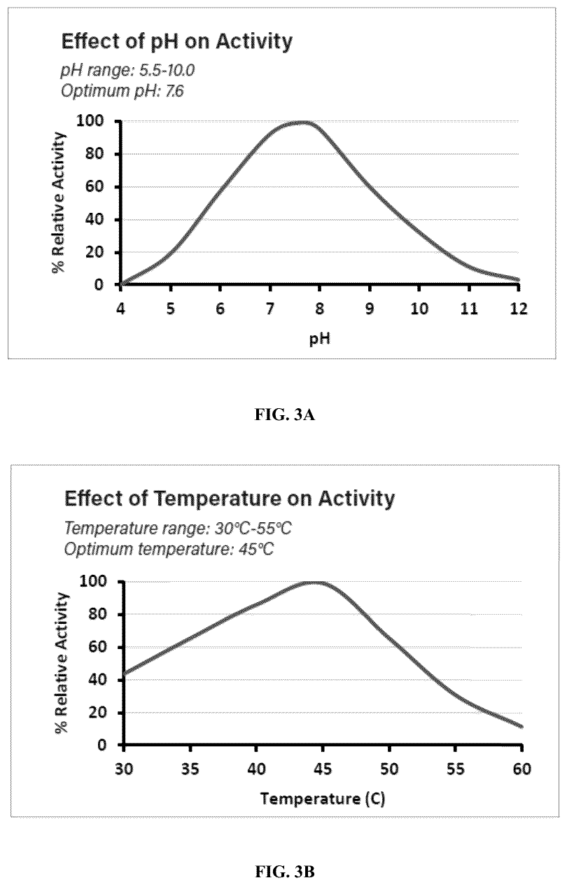

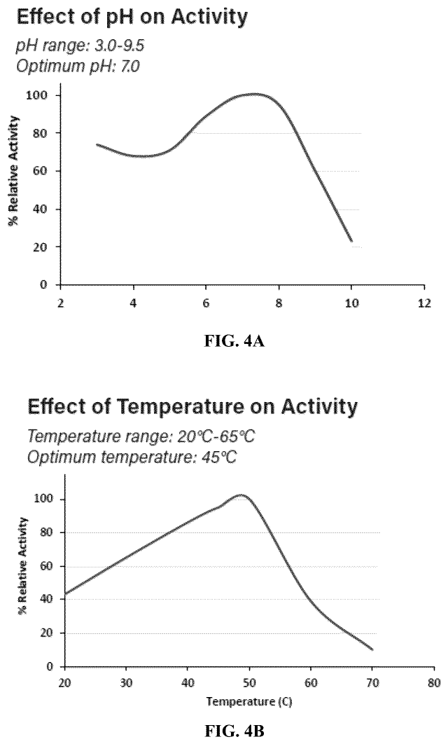

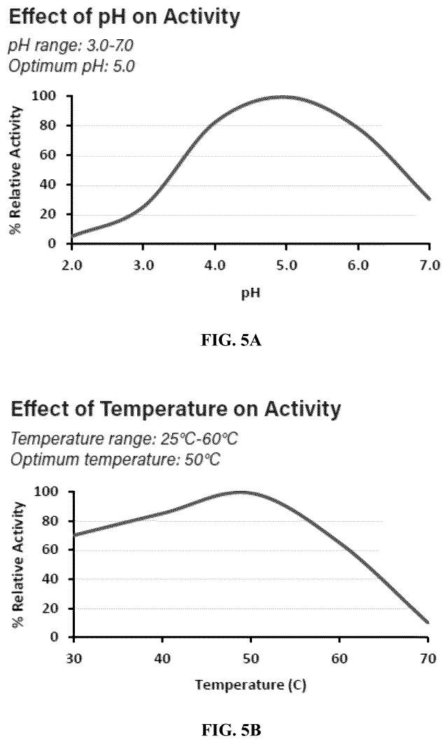

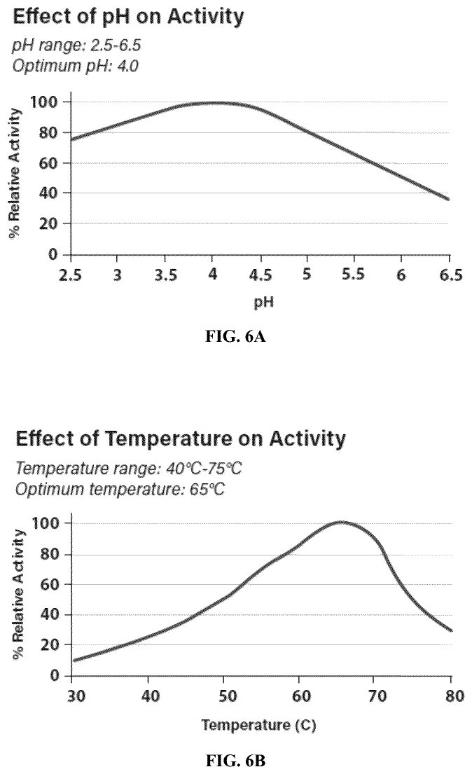

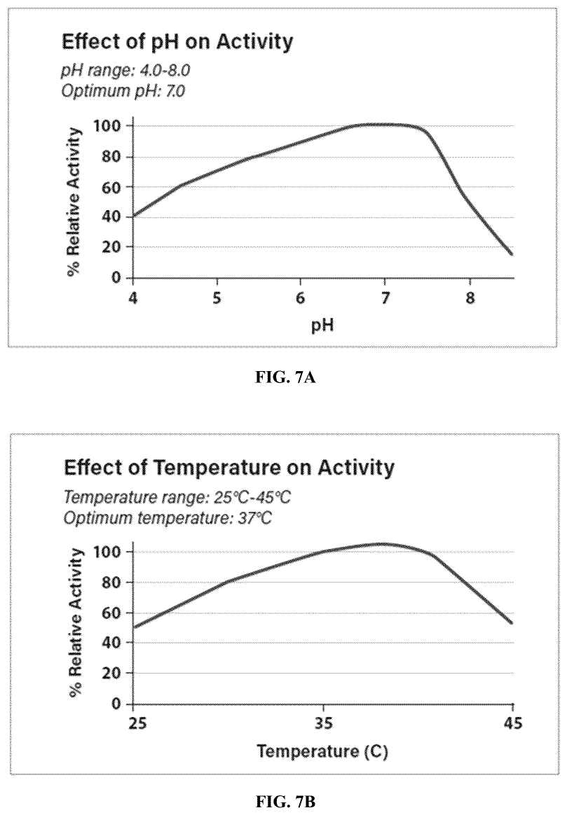

FIGS. 1A-1B provide graphs that illustrate the relative activity levels of Fungal Protease A across various temperature and pH levels. FIGS. 2A-2B provide graphs that illustrate the relative activity levels of Acid Stable Protease A across various temperature and PH levels. FIGS. 3A-3B provide graphs that illustrate the relative and residual activity levels of Protease AM across various temperature and pH levels. FIGS. 4A-4B provide graphs that illustrate the relative activity levels of Yeast Lipase across various temperature and PH levels. FIGS. 5A-5B provide graphs that illustrate the relative activity levels of Fungal Amylase across various temperature and PH levels. FIGS. 6A-6B provide graphs that illustrate the relative activity levels of Glucoamylase across various temperature and PH levels. FIGS. 7A-7B provide graphs that illustrate the relative activity levels of a Rhizopus lipase across various temperature and PH levels.

While the assay results described herein pertain to BC-006 (e.g., Examples 1-3), it is understood that in some alternative aspects, an enzyme mixture according to the disclosure may include at least 4 of the enzymes in BC-006 or comprise at least 4 of SEQ ID Nos: 1, 2, 3, 4, 5, 6, or 7 (e.g., SEQ ID Nos: 1, 2, 3, and 4, or 1, 2, 4, and 5, or 1, 4, 5, and 6, and so forth). It is further understood that the amounts or ratios of the enzymes in the enzyme mixture may be varied to produce a mixture having enhanced or reduced activity levels. For example, Fungal Protease A, Acid Stable Protease A, Fungal Protease AM, Yeast Lipase, Fungal Amylase, and Glucoamylase may be combined at a ratio of approximately 2400:12:2:120:400:1, 1200:4:1:120:400:1, 4000:12:2:20:400:1, 12000:60:10:600:1000:1 as measured in HUT:SAPU:LAPU:FIP:SKB:AGU, or any other ratio which provides a desired activity level as measured in HUT, SAPU, LAPU, FIP, SKB, and AGU units. In some aspects, the ratio of any of the individual components may vary by ±5, 10, 15, 20, 25, 30, 35, 40, 45, or 50% from any of the foregoing examples or ratios described herein. It is further understood that the total HUT activity of the enzyme mixture may be used in place of the three individual protease activity units (i.e., HUT from Fungal Protease A, SAPU from Acid Stable Protease A, LAPU from Protease AM), wherein the ratio of the sum of the 3 fungal proteases: lipase:amylase:glucoamylase is 3600:120:400:1 or 1800-5400:30-180:200-600:0.5-1.5. It is further understood that alpha-amylase activity may alternatively be characterized in dextrinizing units (DU), whereby 1 SKB=1 DU.

In some aspects, the enzyme mixture described herein is dehydrated, powdered, granular, vacuum-dried, or in a freeze-dried form. In a particular aspect, the enzyme mixture described herein is a spray-dried product. Spray drying is a method of producing a dry powder from a liquid or slurry by rapidly drying with a hot gas, and is a preferred method of drying many thermally-sensitive materials, such as foods and pharmaceuticals. In some aspects, the enzyme mixture described herein is a liquid product.

### Dietary Supplements Comprising Enzyme Mixture

In some aspects, enzyme mixtures described herein may be formulated as dietary supplements, protein supplements, and/or nutritional supplement compositions that may be administered to a subject to provide one or more benefits (e.g., to improve protein digestion or the absorption of amino acids, EAAs, and/or BCAAs, to improve fat digestion or the absorption of fatty acids, to improve carbohydrate digestion or the absorption of glucose, to improve postprandial nutrient levels, to improve GI tolerance, to decrease GI symptoms, to improve bowel function, to improve sleep quality, to improve the subject's muscle health, to improve the subject's digestive health, and/or to improve the subject's GI health). As used herein, the term “dietary supplement” refers to a manufactured product taken by mouth that comprises one or more “dietary ingredients” intended to supplement the diet (i.e., food) of a subject. Exemplary dietary ingredients include proteins, amino acids, carbohydrates, fat, vitamins, minerals, metabolites, probiotics, enzymes, herbs and botanicals. Unlike medicaments, dietary supplements are not intended to treat, diagnose, prevent, or cure diseases. A dietary supplement may comprise, e.g., an enzyme mixture as described herein and at least 10, 20, 30, 40, 50, 60, 70, 80, or 90 wt. % of a dietary ingredient or a mixture of dietary ingredients. In some aspects, the dietary ingredient portion comprises a wt. % within a range bounded by any of these values (e.g., 10-20 wt. %). A “protein supplement” is a type of dietary supplement comprising at least 10 dry wt. % protein, wherein the amount of protein in the composition is greater than that of either carbohydrate or fat. A “nutritional supplement” is a type dietary supplement comprising protein, carbohydrates, and fat.

In other aspects, the enzyme mixtures contain at least 50% by weight solids of enzyme preparations from Aspergillus spp., including at least 5%, at least 10%, at least 20%, at least 30%, at least 40%, and at least 50% by weight solids of a protease preparation from A. oryzae, and including at least 5%, at least 10%, at least 20%, at least 30%, at least 40%, and at least 50% by weight solids of a protease preparation from A. niger, and including at least 5%, at least 10%, at least 20%, at least 30%, at least 40%, and at least 50% by weight solids of a protease preparation from A. melleus, and including at least 0.1%, at least 1%, at least 5%, at least 10%, at least 20%, at least 30%, at least 40%, and at least 50% by weight solids of a lipase preparation from Candida cylindracea (or R. oryzae), and including at least 5%, at least 10%, at least 20%, at least 30%, at least 40%, and at least 50% by weight solids of an amylase preparation from A. oryzae, and including at least 1%, at least 5%, at least 10%, at least 20%, at least 30%, at least 40%, and at least 50% by weight solids of a glucoamylase preparation from A. niger. In other aspects, the enzyme mixture contains 30% to 100% total HUT activity from the Aspergillus oryzae protease preparation, including from 35% to 95%, 40% to 90%, 45% to 85%, 50% to 80%, and 55% to 75% total HUT activity of the enzyme mixture. In other aspects, the enzyme mixture contains 30% to 100% total SAPU activity from the Aspergillus niger protease preparation, including from 30% to 100%, 40% to 100%, 50% to 100%, 60% to 100%, and 70% to 100% total SAPU activity of the enzyme mixture. In other aspects, the enzyme mixture contains 30% to 100% total LAPU activity from the Aspergillus melleus protease preparation, including from 30% to 100%, 40% to 100%, 50% to 100%, 60% to 100%, and 70% to 100% total LAPU activity of the enzyme mixture. In other aspects, the enzyme mixture contains 30% to 100% total FIP activity from the Candida cylindracea (or R. oryzae) lipase preparation, including from 30% to 100%, 40% to 100%, 50% to 100%, 60% to 100%, and 70% to 100% total FIP activity of the enzyme mixture. In other aspects, the enzyme mixture contains 30% to 100% total SKB activity from the Aspergillus oryzae amylase preparation, including from 30% to 100%, 40% to 100%, 50% to 100%, 60% to 100%, and 70% to 100% total SKB activity of the enzyme mixture. In other aspects, the enzyme mixture contains 30% to 100% total AGU activity from the Aspergillus niger glucoamylase preparation, including from 30% to 100%, 40% to 100%, 50% to 100%, 60% to 100%, and 70% to 100% total AGU activity of the enzyme mixture.

In some aspects, dietary supplements, protein supplements, and/or nutritional supplement compositions comprising an enzyme mixture according to the disclosure may be suitable for oral administration. Oral administration, as defined herein, includes any form of administration in which the composition including the enzyme mixture passes through the esophagus of the subject. For example, oral administration typically refers to oral consumption, but may also include administration through nasogastric intubation, in which a tube is run from the nose to the stomach of the subject to administer the composition. Oral administration is a form of enteral administration (i.e., administration through the GI tract). Other forms of enteral administration suitable for use with the methods disclosed herein include administration through a gastric or jejunal tube. In other aspects, suitable forms of the composition for enteral administration to the subject include caplets, tablets, pills, capsules, chewable tablets, gummies, quick dissolve tablets, effervescent tablets, solutions, suspensions, emulsions, multi-layer tablets, bi-layer tablets, soft gelatin capsules, hard gelatin capsules, lozenges, chewable lozenges, beads, granules, particles, microparticles, dispersible granules, sachets, and combinations thereof.

In some aspects, dietary supplement, protein supplement, and/or nutritional supplement compositions may be formulated consisting of or consisting essentially of an enzyme mixture according to the disclosure. In other aspects, the enzyme mixture is formulated into a protein supplement. Such protein supplements disclosed herein are useful to provide supplemental sources of protein, including providing the subjects one or more benefits as described herein. In other aspects, the enzyme mixture is formulated in to a nutritional supplement. Such nutritional supplements disclosed herein are useful to provide supplemental sources of nutrition, including providing the subjects one or more benefits as described herein.

In other aspects, the dietary supplement comprises an enzyme mixture according to the disclosure and at least one additional enzyme comprising (a) a lactase; (b) an alpha-galactosidase; (c) a beta-fructofuranosidase; (d) a cellulase; (e) papain; and/or (f) bromelain.

In other aspects, the dietary supplements, protein supplements, and nutritional supplement compositions may be provided as needed to deliver the desired level of an enzyme mixture, e.g., by providing at least one serving per day to achieve the desired effect. In some aspects, the dietary supplements, protein supplements, and nutritional supplement compositions maybe administered at one serving per day, two servings per day, three servings per day, four servings per day, etc., as needed to achieve a desired effect. Typically, the compositions disclosed herein are administered in at least one serving per day or at least two servings per day.

In other aspects, the subject described herein is at least 40 years old, 45 years old, at least 55 years old, at least 60 years old, at least 65 years old, or at least 70 years old.

In another aspect, the subject described herein consumes less than the daily recommended intake of nutrients.

In other aspects, the subject described herein may experience moderate GI symptoms, as measured by an average daily GI score≥1 from the BF-GITFQ (as described herein) or average weekly daily GI score≥1 from the GITQ (as described herein).

In other aspects, the subject described herein may have an average daily stool form score of 1, 2, 6, or 7 based on the Bristol Stool Form Scale.

In another aspect, the subject described herein experiences poor sleep quality.

In some aspects, the enzyme mixture is administered as a dietary supplement in capsule form before, during, or following consumption of a meal (e.g., breakfast, brunch, lunch, dinner, snack). The final dose per serving of the enzyme mixture may be between 5,000 and 500,000 HUT, 10,000 and 200,000 HUT, and 50,000 and 100,000 HUT; between 10 and 1,000 SAPU, 50 and 750 SAPU, and 100 and 500 SAPU; between 1 and 500 LAPU, 5 and 250 LAPU, and 15 and 50 LAPU; between 50 and 20,000 FIP, 100 and 10,000 FIP, and 1,000 and 5,000 FIP; between 500 and 100,000 SKB, 2,000 and 50,000 SKB, and 5,000 and 20,000 SKB; between 1 and 500 AGU, 5 and 250 AGU, and 10 and 50 AGU. Table 1 shows an example formulation, from which a 250 mg capsule would be expected to deliver ˜90,000 HUT (60,000 HUT from A. niger protease), ˜300 SAPU, ˜50 LAPU, 3,000 FIP, 10,000 SKB, and 25 AGU.

In one aspect, an exemplary serving of BC-006 (as described herein) in a dietary supplement will contain 60,000 HUT from Fungal Protease A obtained from A. oryzae, 300 SAPU from Acid Stable Protease A obtained from A. niger, 50 LAPU from Protease AM obtained from A. melleus, 3,000 FIP from Yeast Lipase obtained from C. cylindracea (or R. oryzae), 10,000 SKB (or 10,000 DU) from Fungal Amylase obtained from A. oryzae, and 25 AGU from Glucoamylase obtained from A. niger.

Table 2 shows an alternative exemplary formulation for a 300 mg capsule.

TABLE 1
An exemplary formulation of a dietary supplement comprising
an enzyme mixture according to the disclosure.

| | AMOUNT | ACTIVITY | |
|---|---|---|---|
| INGREDIENTS | (mg/1,000 mg) | (per 1 g) | |
| Maltodextrin | 210 | n/a | |
| Fungal Amylase | 206 | 40,000 | SKB |
| (Aspergillus oryzae) | | | |
| Fungal Protease A | 194 | 240,000 | HUT |
| (Aspergillus oryzae) | | | |
| Acid Stable Protease A | 136 | 1,200 | SAPU |
| (Aspergillus niger) | | | |
| Fungal Protease AM | 128 | 200 | LAPU |
| (Aspergillus melleus) | | | |
| Glucoamylase (Aspergillus niger) | 102 | 100 | AGU |
| Yeast Lipase (Candida cylindracea) | 24 | 12,000 | FIP |

TABLE 2
A second exemplary formulation of a dietary supplement in capsule
form comprising an enzyme mixture according to the disclosure.

| | AMOUNT | ACTIVITY | |
|---|---|---|---|
| | (mg per 300 mg | (per 300 mg | |
| INGREDIENTS | dose) | dose) | |
| Maltodextrin | 94 | n/a | |
| Fungal Amylase | 52 | 10,000 | SKB |
| (Aspergillus oryzae) | | | |
| Fungal Protease A | 48 | 60,000 | HUT |
| (Aspergillus oryzae) | | | |
| Acid Stable Protease A | 35 | 300 | SAPU |
| (Aspergillus niger) | | | |
| Fungal Protease AM | 32 | 50 | LAPU |
| (Aspergillus melleus) | | | |
| Glucoamylase | 26 | 25 | AGU |
| (Aspergillus niger) | | | |
| Yeast Lipase | 7 | 3,000 | FIP |
| (Candida cylindracea) | | | |

| | | |
|---|---|---|
| Magnesium stearate | 5 | n/a |
| Silicon dioxide | 1 | n/a |

In some aspects of the disclosure, the fungal enzyme mixture is administered as a dietary supplement in the form of a powder sachet or stick pack reconstituted in 4 to 6 ounces of water before, during, or following consumption of a meal. The final dose per serving of the enzyme mixture may be between 5,000 and 500,000 HUT, 10,000 and 200,000 HUT, and 50,000 and 100,000 HUT; between 10 and 1,000 SAPU, 50 and 750 SAPU, and 100 and 500 SAPU; between 1 and 500 LAPU, 5 and 250 LAPU, and 15 and 50 LAPU; between 50 and 20,000 FIP, 100 and 10,000 FIP, and 1,000 and 5,000 FIP; between 500 and 100,000 SKB, 2,000 and 50,000 SKB, and 5,000 and 20,000 SKB; between 1 and 500 AGU, 5 and 250 AGU, and 10 and 50 AGU. Table 3 shows an example formulation, from which a 4.5 g stick pack would be expected to deliver ˜90,000 HUT (60,000 HUT from A. niger protease), ˜300 SAPU, ˜50 LAPU, 3,000 FIP, 10,000 SKB, and 25 AGU.

TABLE 3
An exemplary formulation of a dietary supplement
in powder sachet or stick pack form comprising
a protein mixture according to the disclosure.

| | AMOUNT PER |
|---|---|
| INGREDIENTS | SERVING (g) |
| Hydrolyzed Collagen | 3.500 |
| Maltodextrin | 0.255 |
| Fungal Amylase (Aspergillus oryzae) | 0.052 |
| Fungal Protease A (Aspergillus oryzae) | 0.048 |
| Acid Stable Protease A (Aspergillus niger) | 0.035 |
| Fungal Protease AM (Aspergillus melleus) | 0.032 |
| Glucoamylase (Aspergillus niger) | 0.026 |
| Stevia Leaf Extract | 0.030 |
| Strawberry Flavor | 0.015 |
| Yeast Lipase (Candida cylindracea) | 0.007 |

In some aspects of the disclosure, the fungal enzyme mixture is formulated as a protein supplement. The final dose per serving of the enzyme mixture may be between 5,000 and 500,000 HUT, 10,000 and 200,000 HUT, and 50,000 and 100,000 HUT; between 10 and 1,000 SAPU, 50 and 750 SAPU, and 100 and 500 SAPU; between 1 and 500 LAPU, 5 and 250 LAPU, and 15 and 50 LAPU; between 50 and 20,000 FIP, 100 and 10,000 FIP, and 1,000 and 5,000 FIP; between 500 and 100,000 SKB, 2,000 and 50,000 SKB, and 5,000 and 20,000 SKB; between 1 and 500 AGU, 5 and 250 AGU, and 10 and 50 AGU. Tables 4 and 5 show example protein powder formulations, from which a serving would be expected to deliver ˜90,000 HUT (60,000 HUT from A. niger protease), ˜300 SAPU, ˜50 LAPU, 3,000 FIP, 10,000 SKB, and 25 AGU.

TABLE 4
An exemplary formulation of a protein supplement comprising
a protein mixture according to the disclosure.

| | AMOUNT PER |
|---|---|
| INGREDIENTS | SERVING (g) |
| Whey protein concentrate | 20.00 |
| Whey protein isolate | 5.00 |
| Cocoa powder | 2.70 |
| Maltodextrin | 1.15 |
| Natural Flavors | 0.60 |
| Xantham Gum | 0.10 |
| Carageenan | 0.10 |
| Salt | 0.10 |
| Fungal Amylase (Aspergillus oryzae) | 0.052 |
| Fungal Protease A (Aspergillus oryzae) | 0.048 |
| Acid Stable Protease A | 0.035 |
| (Aspergillus niger) | |
| Fungal Protease AM | 0.032 |
| (Aspergillus melleus) | |
| Glucoamylase (Aspergillus niger) | 0.026 |
| Sucralose | 0.030 |
| Acesulfame potassium | 0.025 |
| Yeast Lipase (Candida cylindracea) | 0.007 |

TABLE 5
A second exemplary formulation of a protein supplement comprising
a protein mixture according to the disclosure.

| | AMOUNT PER |
|---|---|
| INGREDIENTS | SERVING (g) |
| Pea Protein Isolate | 11.0 |
| Rice Protein | 6.0 |
| Hemp Protein Isolate | 3.0 |
| Coconut Oil | 2.0 |
| Tapioca Maltodextrin | 1.8 |
| Natural Vanilla Flavors | 0.6 |
| Guar Gum | 0.1 |
| Carageenan | 0.1 |
| Sea Salt | 0.1 |
| Stevia Leaf Extract | 0.08 |
| Fungal Amylase (Aspergillus oryzae) | 0.052 |
| Fungal Protease A (Aspergillus oryzae) | 0.048 |
| Acid Stable Protease A | 0.035 |
| (Aspergillus niger) | |
| Fungal Protease AM | 0.032 |
| (Aspergillus melleus) | |
| Glucoamylase (Aspergillus niger) | 0.026 |
| Yeast Lipase (Candida cylindracea) | 0.007 |

Any source of protein may be used so long as it is suitable for protein supplement or nutritional supplement compositions and is otherwise compatible with any other selected ingredients or features in the protein supplement or nutritional supplement compositions. For example, the source of protein may include, but is not limited to, intact, hydrolyzed, and partially hydrolyzed protein, which may be derived from any known or otherwise suitable source such as milk (e.g., casein, whey), animal (e.g., meat, fish, egg), cereal (e.g., rice, corn, oat, wheat), vegetable (e.g., pea, soy, hemp, potato), pulses (chick pea, mung bean, fava bean), insect and combinations thereof. The source of protein may also include a mixture of amino acids known for use in protein supplements or a combination of such amino acids with the intact, hydrolyzed, and partially hydrolyzed proteins described herein. The amino acids may be naturally occurring or synthetic amino acids. The amino acids may include branched chain amino acids, essential amino acids, non-essential amino acids, or combination thereof.

Examples of suitable sources of protein for use in the protein supplements and nutritional supplements disclosed herein include, but are not limited to, whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, pea protein isolates, pea protein hydrolysates, soy protein concentrates, soy protein isolates, soy protein hydrolysates, pea protein concentrates, collagen proteins, potato proteins, rice proteins, insect proteins, earthworm proteins, fungal (e.g., mushroom) proteins, proteins expressed by microorganisms (e.g., bacteria and algae), and the like, as well as combinations thereof. The nutritional supplement compositions can include any individual source of protein or a combination of two or more the various sources of protein listed above or otherwise encompassed by the general inventive concepts.

A variety of dairy protein and plant protein sources may be utilized for the protein system of the protein supplement or nutritional supplement described herein. An exemplary dairy protein suitable for use in the nutritional supplement powder described herein is Avonlac® 282, a whey protein concentrate, available from Glanbia Nutritionals (Kilkenny, Ireland). An exemplary plant protein suitable for use in the nutritional supplement powder described herein is NUTRALYS* S85F, a pea protein isolate, available from Roquette Frères (Lestrem, France).

In some aspects of the disclosure, the fungal enzyme mixture is formulated into a nutritional supplement. The final dose per serving of the enzyme mixture may be between 5,000 and 500,000 HUT, 10,000 and 200,000 HUT, and 50,000 and 100,000 HUT; between 10 and 1,000 SAPU, 50 and 750 SAPU, and 100 and 500 SAPU; between 1 and 500 LAPU, 5 and 250 LAPU, and 15 and 50 LAPU; between 50 and 20,000 FIP, 100 and 10,000 FIP, and 1,000 and 5,000 FIP; between 500 and 100,000 SKB, 2,000 and 50,000 SKB, and 5,000 and 20,000 SKB; between 1 and 500 AGU, 5 and 250 AGU, and 10 and 50 AGU. Table 6 shows an example nutritional formulation, from which a serving would be expected to deliver ˜90,000 HUT (60,000 HUT from A. niger protease), ˜300 SAPU, ˜50 LAPU, 3,000 FIP, 10,000 SKB, and 25 AGU:

TABLE 6
An exemplary formulation of a nutritional supplement comprising
a protein mixture according to the disclosure.

| | AMOUNT PER |
|---|---|
| INGREDIENTS | SERVING (g) |
| Corn Maltodextrin | 20.0 |
| Sugar | 10.0 |
| Milk protein concentrate | 10.0 |
| Soybean Oil | 5.0 |
| Soy Protein Isolate | 5.0 |
| Canola Oil | 3.0 |
| Vitamin and Mineral Blend | 1.5 |
| Whey Protein Concentrate | 1.0 |
| Guar Gum | 1.0 |
| Carageenan | 1.0 |
| Salt | 0.8 |
| Natural Flavors | 0.6 |
| Fungal Amylase (Aspergillus oryzae) | 0.052 |
| Fungal Protease A (Aspergillus oryzae) | 0.048 |
| Acid Stable Protease A | 0.035 |
| (Aspergillus niger) | |
| Fungal Protease AM | 0.032 |
| (Aspergillus melleus) | |
| Sucralose | 0.030 |
| Glucoamylase (Aspergillus niger) | 0.026 |
| Acesulfame potassium | 0.025 |
| Yeast Lipase (Candida cylindracea) | 0.007 |

The dietary supplements, protein supplements and nutritional supplements described herein may be administered to a subject in order to improve protein digestion or the absorption of amino acids, EAAs, and/or BCAAs, to improve fat digestion or the absorption of fatty acids, to improve carbohydrate digestion or the absorption of glucose, to improve post-prandial nutrient levels, to improve GI tolerance, to decrease GI symptoms, to improve bowel function, to improve sleep quality, to improve the subject's muscle health, to improve the subject's digestive health, and/or to improve the subject's GI health. In each case, such methods comprise administering at least one serving per day of a composition comprising an enzyme mixture according to the disclosure. In some aspects, such methods comprise administering 10 mg to 1,000 mg of enzymes per serving, or approximately 5,000 HUT to 500,000 HUT per serving, and approximately 10 SAPU to 1,000 SAPU per serving, and approximately 10 to 500 LAPU per serving, and approximately 50 to 20,000 FIP per serving, and approximately 500 to 100,000 SKB per serving, and approximately 1 to 500 AGU per serving to the subject.

In other aspects, the dietary supplement comprising the enzyme mixtures described herein may be a powder, such as a powder that may be added to foods or drinks. In some aspects, the dietary supplement comprises the enzyme mixtures described herein and one or more of the following additives: natural or artificial sweeteners (e.g., sugar or sucralose), soluble fiber (e.g., inulin, guar gum, galacto-oligosaccharides, psyllium, pectin), insoluble fiber (e.g., wheat bran), flavoring agents, colorants/dyes, stabilizers, preservatives, anti-caking agents, vitamins, minerals, amino acids, peptides, proteins, botanicals (e.g., ginger extract, green tea extract, cranberry extract, pomegranate powder, beetroot powder, chamomile) probiotics (e.g., strains from the bacterial species Bacillus subtilis, Bacillus coagulans, Bifidobacterium bifidum, Limosilactobacillus reuteri, strains from the bacterial family Bacillaceae, strains from the bacterial phyla Bacillota, Actinomycetota, Bacteroidota, or Pseudomonadota, or strains from the fungal order Saccharomyceses), and combinations thereof.

### Food Products and Beverages Comprising Enzyme Mixture

The enzyme mixtures described herein may be included in a variety of food products and beverages. In some aspects, the composition comprising enzyme mixtures described herein is a food product, such as a baked good (e.g., any baked good that comprises flour). In other aspects, the beverage is a hot beverage (e.g., tea, coffee), while in others it is a cold beverage (juice, soda). The enzyme mixtures described herein may be added to the food or beverage during processing by a manufacturer, or by an end user (e.g., by a consumer adding a dry mixture comprising enzyme mixtures described herein and optionally other nutrients to a water or another liquid to prepare a beverage). In other aspects, the beverage product comprises enzyme mixtures described herein and one or more of the following additives: natural or artificial sweeteners (e.g., sugar or sucralose), soluble fiber (e.g., inulin, guar gum, galacto-oligosaccharides, psyllium, pectin), insoluble fiber (e.g., wheat bran), flavoring agents, colorants/dyes, stabilizers, preservatives, oils (e.g., fatty acids), emulsifiers, vitamins, minerals, amino acids, peptides, and/or proteins.

All statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

### EXAMPLES

The following examples demonstrate the performance of an exemplary enzyme mixture (“BC-006”) comprising Fungal Protease A, Acid Stable Protease A, Protease AM, Yeast Lipase, Fungal Amylase, and Glucoamylase at a ratio 2400:12:2:120:400:1, as measured in HUT:SAPU:LAPU:FIP:SKB:AGU. Each of these enzymes was obtained from BIO-CAT, Inc. (Troy, Virginia, USA).

### Example 1: Performance of BC-006 After In Vitro Gastrointestinal Digestion Simulation—Release of Free Amino Nitrogen and Free Amino Acids

The INFOGEST (INternational Network on FOod DiGESTion) simulation of GI digestion was used to test the efficacy of the BC-006 enzyme mixture on food digestion in vitro. Substrates included each of 1) a canned test meal (CTM) containing canned chicken, canned peas, instant potatoes, water, and unsalted butter; 2) Ensure® oral nutritional supplement powder (ONS); and 3) pea protein isolate powder (PPI). The INFOGEST protocol has been extensively described elsewhere. See Minekus et al., “A Standardised Static In Vitro Digestion Method Suitable for Food —An International Consensus.” Food and Function. 2014. 5(6), 1113-1124; Brodkorb et al., “INFOGEST Static In Vitro Simulation of Gastrointestinal Food Digestion.” Nature Protocols. 2019. 14(4), 991-1014, each of which is hereby incorporated by reference in its entirety). The INFOGEST protocol models three phases of digestion: salivary, gastric, and intestinal. The salivary phase proceeded for 2 minutes in a simulated salivary fluid with agitation at 37° C. and neutral pH in the presence of porcine salivary amylase. The gastric phase proceeded by addition of simulated gastric fluid containing porcine pepsin and incubation for 2 hours with agitation at 37° C. at a starting pH of 3. The intestinal phase proceeded by addition of a simulated intestinal fluid and incubation for an additional 2 hours with agitation at 37°° C. at neutral pH. The simulated intestinal fluid contained porcine pancreatin (mixture of amylases, proteases and lipases from pig) and bile salts. In the experimental groups (“treatments”), a partial dose of BC-006, based on the partial serving size of the food substrate, was added to the gastric digesta 10 minutes after the start of the gastric phase to mimic the time to dissolution of a vegetarian capsule shell in the human stomach. The control groups contained food substrate and the endogenous porcine amylase, porcine pepsin, and pancreatin enzymes in the salivary, gastric, and intestinal phases, respectively, to model human endogenous enzyme activities. Small samples were withdrawn at the end of the 2 hour gastric phase, and the end of the 2 hour intestinal phase, followed by inactivation of enzymatic activity at 90° C. for 10 minutes. Analytical testing included a spectrophotometric method for the determination of free amino nitrogen (FAN) as a marker for protein digestion, a high performance liquid chromatography (HPLC) method for the determination of amino acids, HPLC methods for the determination of glycerol and triglycerides as markers of fat digestion, and HPLC methods for the determination of maltose and glucose as markers for carbohydrate digestion.

Statistical analyses were performed using R® version 3.6.2 (R Core Team, 2020). Figures were produced using GraphPad Prism version 9.1.2 for Windows (San Diego, California, USA). Independent two-tailed t-tests were performed for each substrate to determine significance. Normality was assessed by Shapiro-Wilk test on residuals. Homoscedasticity was assessed with the Levene's Test of Equality of Variances. No violations of normality or homoscedasticity were observed. Figures with asterisks indicate increased levels of significance as follow: *, P<0.05; **, P<0.01; ***, P<0.001; ****, P<0.0001.

In preparation for the in vitro digestion simulation, food substrates and BC-006 enzyme solutions were prepared. 25 grams of the canned chicken test meal (approximately 1/10 serving size) was utilized for each treatment and control. 6 grams of Ensure ONS (approximately 1/10 serving size) was dissolved in 19 mL deionized water for each treatment and control. 2.8 grams of pea protein isolate (approximately 1/10 serving size) was dissolved in 22.2 mL deionized water for each treatment and control. For the experimental treatments, 0.25 grams of BC-006 was weighed on tared weigh paper and then transferred to 20 mL test tubes using 10 mL deionized water. Simulated digestion was initiated per the INFOGEST protocol outline described herein. One mL of each BC-006 solution (approximately 1/10 recommended dose) was added to the gastric digesta 10 minutes after the start of the gastric phase to mimic the dissolution of a vegetarian capsule shell. One mL deionized water (instead of BC-006) was added to the control group. The control group without exogenous enzymes contained substrate and the “endogenous” porcine enzymes amylase and pepsin in the salivary and gastric phases, respectively, to model human endogenous enzyme activities. After the 2 hour gastric phase, a 10 mL sample was pulled into a 15 mL tube and enzymatic activity was halted by placing tubes in a 90° C. water bath for 10 minutes. After the additional 2 hour intestinal phase, another 10 mL sample was pulled into a 15 mL tube and enzymatic activity was halted by placing tubes in a 90° C. water bath for 10 minutes. Samples were stored at 4° C. until spectrophotometric or HPLC analyses. Each experiment was performed in triplicate on separate days.

Following GI simulation, the resulting hydrolysate was collected and analyzed by HPLC using o-phthalaldehyde (OPA) and 9-fluorenylmethyl chloroformate (FMOC). Combining OPA and FMOC enables fast pre-column derivatization of amino acids for chromatographic analysis. The HPLC reaction mixture was buffered at a pH of 10.2, which allows direct derivatization of acid hydrolyzed protein/peptide samples. The primary amino acids were reacted first with OPA using 3-mercaptopropionic acid (3-MPA). The secondary amino acids do not react with the OPA, but are then derivatized using FMOC. The incorporation of 3-MPA into the indoles decreases their hydrophobicity, and as a result, the OPA derivatives elute chromatographically before the FMOC derivatives. Excess FMOC and its degradation products elute after the last of the secondary AAs and do not interfere with the analysis. Samples were measured for protein degradation by FAN analysis using the NOPA method. The amino nitrogen groups of free amino acids in the sample react with N-acetyl-L-cysteine and OPA to form isoindole derivatives. The amount of isoindole derivative formed in the reaction is stoichiometric with the amount of FAN and is measured by the increase in absorbance at 335 nm. To sum, an increase in FAN concentration means that there is an increase in exposed “free amino” peptide ends, suggesting that proteolytic activity has hydrolyzed a larger peptides into smaller peptides (i.e., improved protein digestion). All samples were mixed well before dilution in water. Results are reported in mg of nitrogen/L.

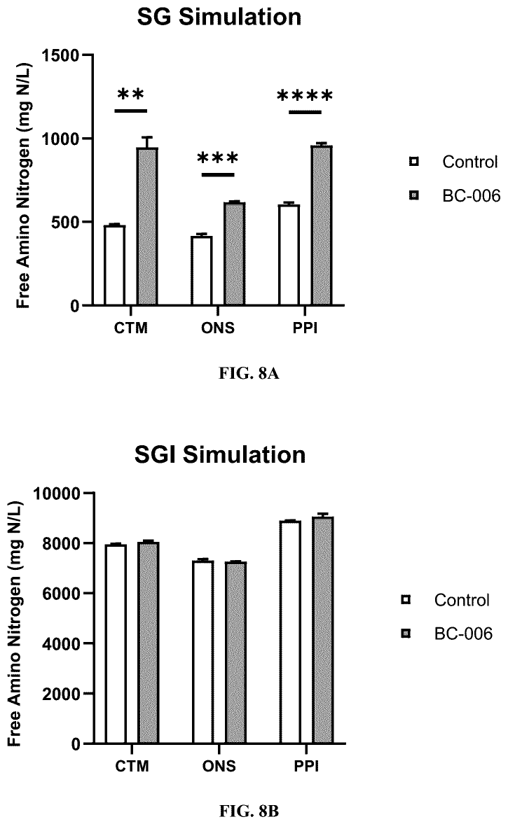

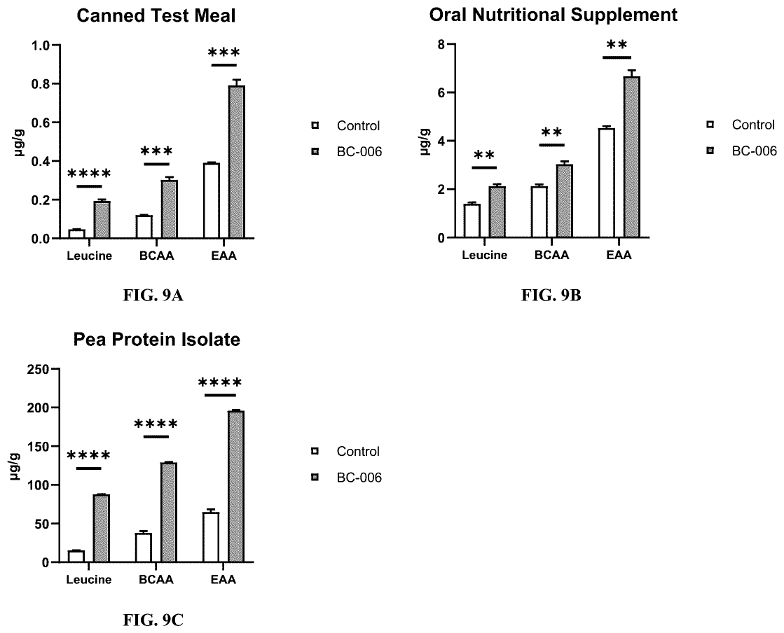

The results of these experiments are summarized by FIGS. 8-9, which provide graphs showing the concentrations of FAN, amino aicds, EAAs, BCAAs and leucine following simulated salivary-gastric (SG) and/or full salivary-gastric-intestinal (SGI) CTM, ONS, and PPI substrates by BC-006, compared to control with same substrate and only the endogenous porcine enzymes. As illustrated by FIG. 8A, in the SG simulation, BC-006 promoted greater FAN release, as compared to control (CTM: P<0.01, ONS: P<0.001, PPI: P<0.0001). In the SGI simulation, no statistically significant differences were observed, illustrated by FIG. 8B. As illustrated by FIGS. 9A-9C, BC-006 promoted greater leucine release, BCAA release, and EAA release, as compared to control. BC-006 promoted greater release of leucine (P<0.0001), BCAA (P<0.001), and EAA (P<0.001) from CTM, greater release of leucine, BCAA, and EAA from ONS (P<0.01), and greater release of leucine, BCAA, and EAA from PPI (P<0.0001), as compared respectively to controls.

In this example, a canned chicken test meal, Ensure® oral nutritional supplement, and pea protein isolate were assayed as protein sources for digestion. However, it is understood that other protein sources obtained from other animals, insects, plants, fungi, or bacteria may also be digested using the enzyme mixtures disclosed herein. Similarly, the incubation time and temperature parameters described above may vary as necessary for a given application, while remaining in accordance with the present disclosure.

Normally, it takes approximately 1-3 hours to reach maximum total serum amino acids levels after protein consumption. As evidenced by these data, BC-006 may be applied to (or ingested concurrently with) a meal, food product, or dietary supplement containing protein to expedite the digestive process and to increase the amount of free amino acids that are available for absorption during digestion. Under in vivo conditions, it is expected that this increase in the amount of free amino acids will result in increased uptake and higher blood amino acid levels and greater bioavailability to skeletal muscle to support muscle health

### Example 2: Performance of BC-006 After In Vitro Gastrointestinal Digestion Simulation—Release of Glycerol and Triglycerides

Following the same protocol described in Example 1, BC-006 was assayed to measure its ability to release glycerol and reduce triglycerides from several dietary substrates.

Glycerol was evaluated by HPLC using a SUPELCOGEL C-610H 30 cm×7.8 mm column kept at 30° C., a flow rate of 0.5 mL/min of 0.1% phosphoric acid, and refractive index detection. Standards and samples were prepared in water. All samples were mixed well and filtered through a 0.45 μm syringe filter prior to dilution in water. Results are reported in mg glycerol/g. Lipid degradation was determined using HPLC to measure triglycerides. This method uses two Supelcosil LC-18 Columns (150×4.6 mm) in series, a flow rate of 1 mL/min of 64:36 acetone:acetonitrile, and refractive index detection. Oil standards and samples were prepared in acetone. Results are reported in mg total triglycerides/mL.

A statistical analysis was performed using R® version 3.6.2 (R Core Team, 2020). Figures were produced using GraphPad Prism version 9.1.2 for Windows (San Diego, California USA). Independent one-tailed t-tests were performed for each substrate to determine significance. Normality was assessed by Shapiro-Wilk test on residuals. Homoscedasticity was assessed with the Levene's Test of Equality of Variances. No violations of normality or homoscedasticity were observed. Figures with asterisks indicate increased levels of significance as follow: *, P<0.05; **, P<0.01; ***, P<0.001; ****, P<0.0001.

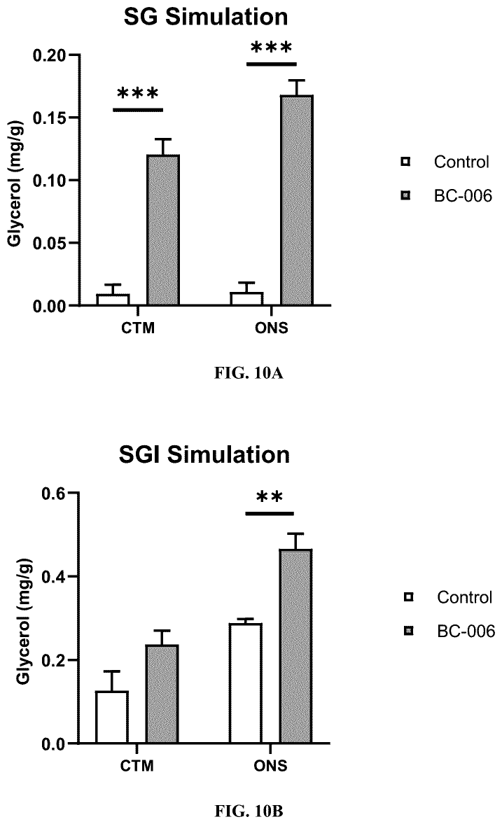

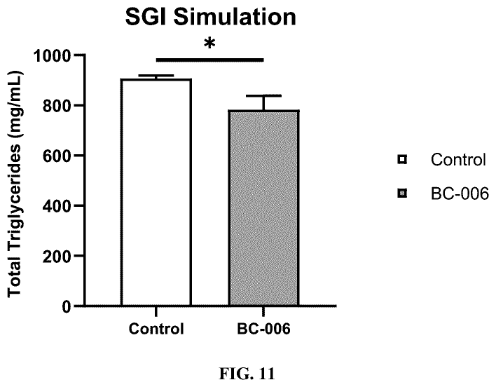

The results of these experiments are summarized by FIGS. 10-11, which provide graphs showing the concentrations of glycerol and triglycerides following simulated gastric and/or full GI digestion of the CTM and ONS substrates by BC-006, compared to control with same substrate and only the endogenous porcine enzymes. As illustrated by FIG. 10A, BC-006 promoted greater glycerol release from the CTM (P<0.001) and ONS (P<0.001) as compared to controls in the SG simulation, but only from ONS with statistical significance (P<0.01) in the SGI simulation. As illustrated in FIG. 10B, BC-006 promoted greater glycerol release from ONS (P<0.01) as compared to controls in the SGI simulation, and approached a statistically significant difference from controls from the CTM (P=0.061). As illustrated by FIG. 11, BC-006 promoted greater triglyceride reduction from unsalted butter following the full GI simulation, as compared to control (P=0.045) Results from these SGI simulations were unexpected, since the amount of porcine pancreatin across treatments and control in the INFOGEST protocol is expected to adequately hydrolyze triglycerides. In such a way, BC-006 and lipase activities therein, showed superior performance on glycerol release and triglyceride reduction following the full SGI simulation.

### Example 3: Performance of BC-006 After In Vitro Gastrointestinal Digestion Simulation—Release of Maltose and Glucose

Following the same protocol described in Example 1, BC-006 was assayed to measure its ability to release maltose and glucose from several dietary substrates.

Carbohydrate degradation was evaluated using HPLC to measure maltose and glucose released from starch. This method uses a SUPELCOGEL C-610H 30 cm×7.8 mm column kept at 30° C., a flow rate of 0.5 mL/min of 0.1% phosphoric acid, and refractive index detection. Standards and samples were prepared in water. All samples were mixed well and filtered through a 0.45 μm syringe filter prior to dilution in water. Results are reported in mg/g.

Following the same statistical procedures described in Example 1, BC-006′s efficacy was tested by its ability to release maltose and glucose from several dietary substrates.

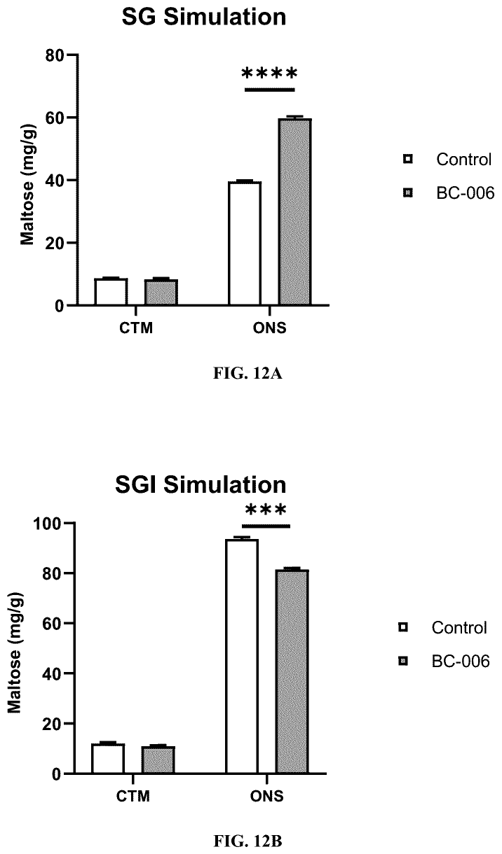

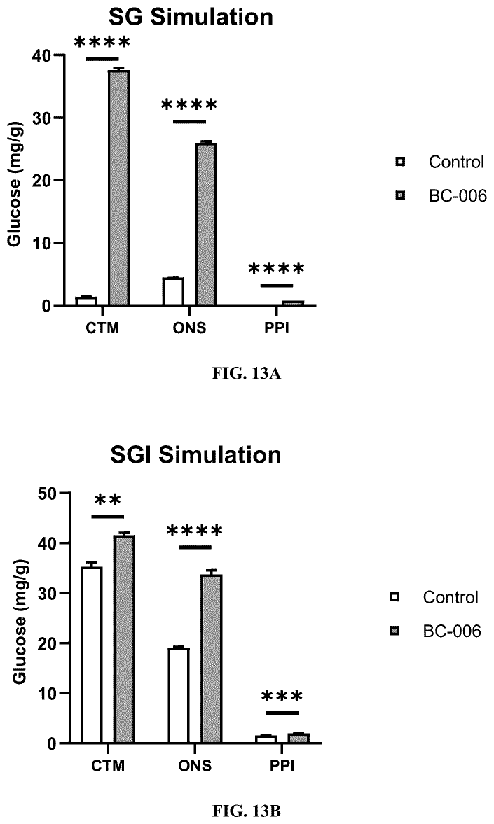

The results of these are summarized by FIGS. 12-13, which provide graphs showing the concentrations of maltose and glucose following simulated gastric and/or full GI digestion of the CTM, ONS, and PPI substrates by BC-006, compared to control with same substrate and only the endogenous porcine enzymes. As illustrated by FIGS. 12A-12B, BC-006 promoted greater maltose release from ONS (P<0.0001) as compared to control with pepsin alone in the SG simulation. BC-006 treatment resulted in lower maltose concentrations following the full SGI simulation of ONS digestion (P<0.001), likely owing to the supplemental glucoamylase activity of BC-006 which hydrolyzes maltose to glucose, resulting in lower maltose. No significant differences in maltose concentrations were observed between BC-006 and control groups following either of SG or SGI simulations of CTM digestion. These data are likely explained by maltose's conversion to glucose by BC-006, as illustrated by FIGS. 13A-13B, showing that BC-006 promoted greater glucose release from the CTM, ONS, and PPI as compared to controls (P<0.0001) in the SG simulation, and from the CTM (P<0.01), ONS (P<0.0001) and PPI (P<0.001) in the SGI simulation. Results from these SGI simulations were unexpected, since the amount of porcine pancreatin across treatments and control in the INFOGEST protocol is expected to adequately hydrolyze starch and maltose. In such a way, BC-006 and amylase and glucoamylase activities therein, showed superior performance on maltose and glucose release following the full SGI simulation.

### Example 4: Clinical Evaluation of the Effects of Enzyme Supplementation on Post-Prandial Blood Amino Acid, Fatty Acid, Glucose, and Iron Levels

A randomized, double-blind, placebo-controlled, crossover design may be used to evaluate the effects of enzyme supplementation using the enzyme mixture described herein. For example, qualified participants may be randomized into groups given either BC-006 (n=15) or placebo (PL; n=15) in conjunction with a standardized test meal for their 1^{st} aminoacidemia trial. Given the nature of the crossover design, a 2^{nd} trial consists of the opposite study product (e.g., if a participant receives BC-006 during the 1^{st} trial, then they will receive placebo during the second trial). The objective of this study would be to determine whether BC-006 enzyme supplementation (e.g., with ˜60,000 HUT activity from Fungal Protease A, ˜300 SAPU activity from Acid Stable Protease A, ˜50 LAPU activity from Protease AM, ˜3,000 FIP activity from Yeast Lipase, ˜10,000 SKB activity from Fungal Amylase, and ˜25 AGU activity from Glucoamylase) is an effective dosage to improve the early (0-3 hour) and cumulative (0-5 hour) incremental area-under-the-curve plasma concentrations, and maximal plasma concentrations of all amino acids, all EAAs, all BCAAs, leucine, fatty acid, glucose, and iron after the ingestion of a standardized test meal in healthy, middle-aged and older subjects (between the ages of 45 and 70 years). Study participants may be asked to report to the testing facility at ˜0700 hours after an overnight fast and undergo catheter placement in an antecubital vein. After taking a baseline blood sample for determination of plasma amino acids, insulin, fatty acids, glucose, and iron, a mixed meal tolerance test may be administered. The mixed meal may comprise 75 g grilled chicken breast strips, 200 g mashed potatoes, 21.3 g unsalted butter, and 120 g steamed green peas, and be administered to subjects within 15 minutes of the baseline blood draw. The test articles may be manufactured in capsule form (e.g., as: 213 mg enzyme blend, 67 mg maltodextrin, 5 mg magnesium stearate, and 1 mg silicon dioxide, or placebo with 280 mg maltodextrin, 5 mg magnesium stearate, and 1 mg silicon dioxide). Participants may be direct to consume study products in between the second and third bites of the standardized test meal. Blood samples may be collected at one or more time points postprandially (e.g., a total of 13 blood samples may be collected 5 hours postprandially, with blood draws at baseline and 30 minutes, 60 minutes, 90 minutes, 105 minutes, 120 minutes, 135 minutes, 150 minutes, 165 minutes, 180 minutes, 210 minutes, and 240 minutes thereafter, with the final blood draw taken 5 hours after meal consumption). Plasma amino acid concentrations may be determined via liquid chromatography with tandem mass spectrometry. Plasma fatty acid concentrations may be determined by gas chromatography-mass spectrometry. Plasma glucose concentrations may be analyzed using an automated glucose analyzer (e.g., YSI 2300 Stat Plus, Yellow Springs Instruments, USA). Serum iron concentrations may be analyzed by spectrophotometry. A study according to this exemplary protocol may be used to evaluate the effects of enzyme supplementation using the enzyme mixture described herein and to provide data that can be used to select optimal amounts and/or administration schedules for the enzyme mixture described herein.

### Example 5: Clinical Evaluation of the Effects of Enzyme Supplementation on Gastrointestinal Tolerance, Bowel Function, and Sleep Quality

A randomized, double-blind, placebo-controlled, crossover design may be used to evaluate the effects of enzyme supplementation using the enzyme mixture described herein. For example, qualified participants may be randomized into groups given either BC-006 (n=15) or placebo (PL; n=15) and directed to consume the study product twice daily with their two largest meals of the day for 21 days. Given the nature of the crossover design, a 2^{nd} phase consists of the same 21-day protocol however with the opposite study product (e.g., if a participant receives BC-006 during phase 1, then they will receive placebo during phase 2). The objective of this study would be to determine whether BC-006 enzyme supplementation (e.g., with ˜60,000 HUT activity from Fungal Protease A, ˜300 SAPU activity from Acid Stable Protease A, ˜50 LAPU activity from Protease AM, ˜3,000 FIP activity from Yeast Lipase, ˜10,000 SKB activity from Fungal Amylase, and ˜25 AGU activity from Glucoamylase) is an effective dosage to improve GI tolerance, to decrease GI symptoms, to improve bowel function, to improve sleep quality, to improve the subject's digestive health, and/or to improve the subject's GI health in healthy, middle-aged and older subjects (e.g., between the ages of 45 and 70 years).

GI tolerance and bowel function may be assessed by provision of a paper Bowel Function and GI Tolerance Factors questionnaire (BF-GITFQ) that is completed by participants daily for the 21 days of the 1^{st} phase of the trial and the following 21 days of the 2^{nd} phase of the trial. GI factors, including burping, cramping/pain, distension/bloating, flatulence/gas, nausea, reflux (heartburn), and rumblings, may be recorded daily on each day of the two successive 21-day trials. Subjects may be asked a series of questions regarding the presence and severity of the GI factors occurring during the past 24 hours, in which each response is ranked on a 4-point scale ranging from none to severe: 0=absent, 1=mild, 2=moderate; 3=severe. GI factors may be assessed individually, or a composite GI symptom score can be calculated as the sum of the ratings of the individual symptoms. Therefore, the total composite GITF symptom score for each day may range from 0 to 21. This daily scale of GI factors has been previously employed in clinical research to assess GI health. See e.g., Holscher et al., “Gastrointestinal Tolerance and Utilization of Agave Inulin by Healthy Adults.” Food and Function. 2014. 5(6), 1142-1149.

Subjects may also be instructed to complete the BF-GITFQ questionnaire after bowel movement regarding the bowel movement frequency, ease of passage, and consistency using the Bristol Stool Form Scale (BSFS). The BSFS was developed and first published by clinicians at the Bristol Royal Infirmary at the University of Bristol (Bristol, United Kingdom). See O'Donnell et al., “Detection of Pseudodiarrhoea by Simple Clinical Assessment of Intestinal Transit Rate.” British Medical Journal (BMJ). 1990. 300(6722), 439-440. The authors described a 7-point scale that characterized stool form based on cohesion and surface cracking: 1, separate hard lumps; 2, sausage shaped but lumpy; 3, like a sausage or snake but with cracks on its surface; 4, like a sausage or snake, smooth and soft; 5, soft blobs with clear cut edges; 6, fluffy pieces with ragged edges, a mushy stool; 7, watery, no solid pieces. Type 1 and 2 stools are considered to be abnormally hard stools and potentially reflective of constipation. Type 6 and 7 stools are considered abnormally loose stools and potentially reflective of diarrhea. Type 3, 4, and 5 stools are considered to be healthy stool forms. Additionally, O'Donnell et al. reported that mean stool form score across 30 patients with Inflammatory bowel syndrome was inversely proportional to whole GI transit time (R=−0.77, P<0.001). In sum, the looser the stool, the quicker the GI transit time. The firmer the stool, the slower the GI transit time. Again, GI transit times associated with Type 3, 4, and 5 stools are considered to be healthy GI transit times. The BSFS has been widely used to measure GI health and disease severity in clinical studies, as well as being validated in larger, healthy subject populations. See e.g., Blake et al., “Validity and Reliability of the Bristol Stool Form Scale in Healthy Adults and Patients with Diarrhoea-Predominant Irritable Bowel Syndrome.” Alimentary Pharmacology and Therapeutics. 2016. 44(7), 693-703; Lacy t al., “Bowel Disorders.” Gastroenterology. 2016. 150, 1393-1407, each of which is hereby incorporated by reference in its entirety). Additionally, a 5-point scale rating ease of passage may be used to assess bowel function. Ease of passage may be scaled 1 to 5:1=very easy; 2=easy; 3 =neither easy nor difficult; 4=difficult; 5=very difficult. Such a scale has been used in previous clinical studies. See e.g., Holscher et al., “Gastrointestinal Tolerance and Utilization of Agave Inulin by Healthy Adults.” Food and Function. 2014. 5 (6), 1142-1149.

GI tolerance may also be assessed by provision of a paper GI Tolerance Questionnaire (GITQ) to be completed by participants weekly on each week of the two 21 day trials. GI symptoms, including nausea, bloating, stomach rumblings, gas/flatulence, abdominal pain, and diarrhea, may be recorded weekly on each week of the two 21-day trials. Subjects may be asked a series of questions regarding the amount of GI symptoms occurring during the past 7 days, in which each response is ranked on a 3-point scale: 0=did not experience, or no more than usual, 1=somewhat more than usual, 2=much more than usual. GI symptoms may be assessed individually, or a composite GITQ symptom score can be calculated as the sum of the ratings of the individual symptoms. Therefore, the total composite GITQ symptom score for each week may range from 0 to 12. This weekly scale of GI symptoms has been previously employed in clinical research to assess GI health. See e.g., Maki et al., “Fibermalt is Well Tolerated in Healthy Men and Women at Intakes Up to 60 g/d: A Randomized, Double-blind, Crossover Trial.” International Journal of Food Sciences and Nutrition. 2013. 64(3), 274-281.

Sleep quality may be assessed by provision of a paper Single-Item Sleep Quality Scale (SI-SQS) survey to be completed by participants weekly on each week of the two 21 day trials. See Snyder et al., “A New Single-Item Sleep Quality Scale: Results of Psychometric Evaluation in Patients With Chronic Primary Insomnia and Depression.” Journal of Clinical Sleep Medicine. 2018. 14(11), 1849-1857. The SI-SQS may direct the subject to rate the overall quality of sleep over a 7-day recall period on a visual analog scale (VAS). The subject may mark an integer score from 0 to 10, according to the following five categories: 0=terrible, 1-3=poor, 4-6=fair, 7-9=good, and 10=excellent. When rating their sleep quality, subjects may be instructed to consider the following core components of sleep quality: how many hours of sleep they had, how easily they fell asleep, how often they woke up during the night (except to go to the bathroom), how often they woke up earlier than they had to in the morning, and how refreshing their sleep was.

A study according to this exemplary protocol may be used to evaluate the effects of enzyme supplementation using the enzyme mixture described herein and to provide data that can be used to select optimal amounts and/or administration schedules for the enzyme mixture described herein.

### DESCRIPTION OF SEQUENCES

| SEQ ID NO: | 1 |
|---|---|
| Trade Name: | Fungal Protease A (BIO-CAT, Inc.) |
| Source organism: | Aspergillus oryzae |
| Protein: | Aspergillopepsin-1 |
| Gene: | pepA |
| Synonyms: | Aspartic protease 2, Aspergillopepsin I, Aspergillopeptidase |
| | A |
| IUBMB Enzyme | 3.4.23.18 |
| Commission (EC) No: | |
| Length: | ≤404 amino acids |
| Amino Acid Sequence: | 10 20 30 40 50 |
| | MVILSKVAAV AVGLSTVASA LPTGPSHSPH ARRGFTINQI TRQTARVGPK |
| | 60 70 80 90 100 |
| | TASFPAIYSR ALAKYGGTVP AHLKSAVASG HGTVVTSPEP NDIEYLTPVN |
| | 110 120 130 140 150 |
| | IGGTTLNLDF DTGSADLWVF SEELPKSEQT GHDVYKPSGN ASKIAGASWD |
| | 160 170 180 190 200 |
| | ISYGDGSSAS GDVYQDTVTV GGVTAQGQAV EAASKISDQF VQDKNNDGLL |
| | 210 220 230 240 250 |
| | GLAFSSINTV KPKPQTTFFD TVKDQLDAPL FAVTLKYHAP GSYDFGFIDK |
| | 260 270 280 290 300 |
| | SKFTGELAYA DVDDSQGFWQ FTADGYSVGK GDAQKAPITG IADTGTTLVM |
| | 310 320 330 340 350 |
| | LDDEIVDAYY KQVQGAKNDA SAGGYVFPCE TELPEFTVVI GSYNAVIPGK |
| | 360 370 380 390 400 |
| | HINYAPLQEG SSTCVGGIQS NSGLGLSILG DVFLKSQYVV FDSQGPRLGF |
| | AAQA |
| Predicted Molecular Weight: | 42313.08 Daltons (irrespective of post-translational |
| | modifications (e.g., signal peptide cleavage, glycosylation, |
| | etc.) and lower molecular weight variants and products) |

| SEQ ID NO: | 2 |
|---|---|
| Trade Name: | Acid Stable Protease A (BIO-CAT, Inc.) |
| Source organism: | Aspergillus niger |
| Protein: | Aspergillopepsin I |
| Gene: | pepA |
| Synonyms: | Aspartic protease pepA, Aspergillopepsin I, |
| | Aspergillopeptidase A |
| IUBMB Enzyme | 3.4.23.18 |
| Commission (EC) No.: | |
| Length: | ≤394 amino acids |
| Amino Acid Sequence: | 10 20 30 40 50 |
| | MVVFSKTAAL VLGLSSAVSA APAPTRKGFT INQIARPANK TRTINLPGMY |
| | 60 70 80 90 100 |
| | ARSLAKFGGT VPQSVKEAAS KGSAVTTPQN NDEEYLTPVT VGKSTLHLDF |
| | 110 120 130 140 150 |
| | DTGSADLWVF SDELPSSEQT GHDLYTPSSS ATKLSGYTWD ISYGDGSSAS |
| | 160 170 180 190 200 |
| | GDVYRDTVTV GGVTTNKQAV EAASKISSEF VQNTANDGLL GLAFSSINTV |
| | 210 220 230 240 250 |
| | QPKAQTTFFD TVKSQLDSPL FAVQLKHDAP GVYDFGYIDD SKYTGSITYT |
| | 260 270 280 290 300 |
| | DADSSQGYWG FSTDGYSIGD GSSSSSGFSA IADTGTTLIL LDDEIVSAYY |
| | 310 320 330 340 350 |
| | EQVSGAQESE EAGGYVFSCS TNPPDFTVVI GDYKAVVPGK YINYAPISTG |
| | 360 370 380 390 |
| | SSTCFGGIQS NSGLGLSILG DVFLKSQYVV FNSEGPKLGF AAQA |
| Predicted Molecular | 41276.42 Daltons (irrespective of post-translational |
| Weight: | modifications (e.g., signal peptide cleavage, glycosylation, |
| | etc.) and lower molecular weight variants and products) |

| SEQ ID NO: | 3 |
|---|---|
| Trade Name: | Fungal Protease AM (BIO-CAT, Inc.) |
| Source organism: | Aspergillus melleus |
| Protein: | Leucine Aminopeptidase 1 |
| Gene: | lap1 |
| IUBMB Enzyme | 3.4.11.- |
| Commission (EC) No.: | |
| Length: | ≤378 amino acids |
| Amino Acid | 10 20 30 40 50 |
| sequence: | MRFPIYATAL AATASALVIG ERASSEDRYI LELAPGVTKV VTEAEKWSLK |
| | 60 70 80 90 100 |
| | AEGKRFFDIT DEVSTIKTAN VEDSQQQLAV TYPSKVQYKD TVEKLIPQLS |
| | 110 120 130 140 150 |
| | KANFQTVLKP FSGFHNRYYK SNYGKQSSEW LQAQIQSVVD ASGAKGITVK |
| | 160 170 180 190 200 |
| | PFSHSWTQSS IIATIPGKSN KIVVLGAHQD SINQASPSTG RAPGADDDGS |
| | 210 220 230 240 250 |
| | GVVTILETLR VLLKDPKVAA GEATNTIEFH FYSAEEGGLL GSQAIFRQYS |
| | 260 270 280 290 300 |
| | SQSKQVVAML QQDMTGYTEG TKKAGKPEAI GVLTDYVDAG LTKFLKTIID |
| | 310 320 330 340 350 |
| | TYTTISWVES QCGYACSDHA SANRYGYPSS FAFESEFGDD SPYIHTAQDT |
| | 360 370 |
| | ISTVDFDHVL QHARLSLGFA YELGFASL |
| Predicted Molecular | 41176.11 Daltons (irrespective of post-translational modifications |
| Weight: | (e.g., signal peptide cleavage, glycosylation, etc.) and lower |
| | molecular weight variants and products) |

| SEQ ID NO: | 4 |
|---|---|
| Trade Name: | Fungal Amylase (BIO-CAT, Inc.) |
| Source organism: | Aspergillus oryzae |
| IUBMB Enzyme | 3.2.1.1 |
| Commission (EC) No .: | |
| Protein: | Alpha-amylase A type-1/2 |
| Gene: | amy1; amy2 |
| Synonyms: | 1,4-alpha-D-glucan glucanohydrolase, Taka-amylase A |
| Length: | ≤499 amino acids |
| Amino Acid Sequence: | 10 20 30 40 50 |
| | MMVAWWSLFL YGLQVAAPAL AATPADWRSQ SIYFLLTDRF ARTDGSTTAT |
| | 60 70 80 90 100 |
| | CNTADRKYCG GTWQGIIDKL DYIQGMGFTA IWITPVTAQL PQTTAYGDAY |
| | 110 120 130 140 150 |
| | HGYWQQDIYS LNENYGTADD LKALSSALHE RGMYLMVDVV ANHMGYDGAG |
| | 160 170 180 190 200 |
| | SSVDYSVFKP FSSQDYFHPF CLIQNYEDQT QVEDCWLGDN TVSLPDLDTT |
| | 210 220 230 240 250 |
| | KDVVKNEWYD WVGSLVSNYS IDGLRIDTVK HVQKDFWPGY NKAAGVYCIG |
| | 260 270 280 290 300 |
| | EVLDGDPAYT CPYQNVMDGV LNYPIYYPLL NAFKSTSGSM DDLYNMINTV |
| | 310 320 330 340 350 |
| | KSDCPDSTLL GTFVENHDNP RFASYINDIA LAKNVAAFII LNDGIPIIYA |
| | 360 370 380 390 400 |
| | GQEQHYAGGN DPANREATWL SGYPTDSELY KLIASANAIR NYAISKDTGF |
| | 410 420 430 440 450 |
| | VTYKNWPIYK DDTTIAMRKG TDGSQIVTIL SNKGASGDSY TLSLSGAGYT |
| | 460 470 480 490 |
| | AGQQLTEVIG CTTVTVGSDG NVPVPMAGGL PRVLYPTEKL AGSKICSSS |
| Predicted Molecular Weight: | 54804.23 Daltons (irrespective of post-translational |
| | modifications (e.g., signal peptide cleavage, glycosylation, |
| | etc.) and lower molecular weight variants and products) |

| SEQ ID NO: | 5 |
|---|---|
| Trade Name: | Yeast Lipase (BIO-CAT, Inc.) |
| Source organism: | Candida cylindraceae (formerly Candida rugosa, Diutina |
| | rugosa) |
| IUBMB Enzyme | 3.1.1.3 |
| Commission (EC) No.: | |
| Protein: | Lipase 1 |
| Gene: | lip1 |
| Length: | ≤549 amino acids |
| Amino Acid Sequence: | 10 20 30 40 50 |
| | MELALALSLI ASVAAAPTAT LANGDTITGL NAIINEAFLG IPFAEPPVGN |
| | 60 70 80 90 100 |
| | LRFKDPVPYS GSLDGQKFTS YGPSCMQQNP EGTYEENLPK AALDLVMQSK |
| | 110 120 130 140 150 |
| | VFEAVSPSSE DCLTINVVRP PGTKAGANLP VMLWIFGGGF EVGGTSTFPP |
| | 160 170 180 190 200 |
| | AQMITKSIAM GKPIIHVSVN YRVSSWGFLA GDEIKAEGSA NAGLKDQRLG |
| | 210 220 230 240 250 |
| | MQWVADNIAA FGGDPTKVTI FGESAGSMSV MCHILWNDGD NTYKGKPLER |
| | 260 270 280 290 300 |
| | AGIMQSGAMV PSDAVDGIYG NEIFDLLASN AGCGSASDKL ACLRGVSSDT |
| | 310 320 330 340 350 |
| | LEDATNNTPG FLAYSSLRLS YLPRPDGVNI TDDMYALVRE GKYANIPVII |
| | 360 370 380 390 400 |
| | GDQNDEGTFF GTSSLNVTTD AQAREYFKQS FVHASDAEID TLMTAYPGDI |
| | 410 420 430 440 450 |
| | TQGSPFDTGI LNALTPQFKR ISAVLGDLGF TLARRYFLNH YTGGTKYSFL |
| | 460 470 480 490 500 |
| | SKQLSGLPVL GTFHSNDIVF QDYLLGSGSL IYNNAFIAFA TDLDPNTAGL |
| | 510 520 530 540 |
| | LVKWPEYTSS SQSGNNLMMI NALGLYTGKD NFRTAGYDAL FSNPPSFFV |
| Predicted Molecular Weight: | 58550.09 Daltons (irrespective of post-translational |
| | modifications (e.g., signal peptide cleavage, glycosylation, |
| | etc.) and lower molecular weight variants and products) |

| SEQ ID NO: | 6 |
|---|---|
| Trade Name: | Glucoamylase (BIO-CAT, Inc.) |
| Source organism: | Aspergillus niger |
| Protein: | Glucoamylase |
| Synonyms: | 1,4-alpha-D-glucan glucohydrolase, glucan 1,4-alpha- |
| | glucosidase |
| IUBMB Enzyme | 3.4.23.18 |
| Commission (EC) No: | |
| Length: | ≤640 amino acids |
| Amino Acid sequence: | 10 20 30 40 50 |
| | MSFRSLLALS GLVCTGLANV ISKRATLDSW LSNEATVART AILNNIGADG |
| | 60 70 80 90 100 |
| | AWVSGADSGI VVASPSTDNP DYFYTWTRDS GLVLKTLVDL FRNGDTSLLS |
| | 110 120 130 140 150 |
| | TIENYISAQA IVQGISNPSG DLSSGAGLGE PKFNVDETAY TGSWGRPQRD |
| | 160 170 180 190 200 |
| | GPALRATAMI GFGQWLLDNG YTSTATDIVW PLVRNDLSYV AQYWNQTGYD |
| | 210 220 230 240 250 |
| | LWEEVNGSSF FTIAVQHRAL VEGSAFATAV GSSCSWCDSQ APEILCYLQS |
| | 260 270 280 290 300 |
| | FWTGSFILAN FDSSRSGKDA NTLLGSIHTF DPEAACDDST FQPCSPRALA |
| | 310 320 330 340 350 |
| | NHKEVVDSFR SIYTLNDGLS DSEAVAVGRY PEDTYYNGNP WFLCTLAAAE |
| | 360 370 380 390 400 |
| | QLYDALYQWD KQGSLEVTDV SLDFFKALYS DAATGTYSSS SSTYSSIVDA |
| | 410 420 430 440 450 |
| | VKTFADGFVS IVETHAASNG SMSEQYDKSD GEQLSARDLT WSYAALLTAN |
| | 460 470 480 490 500 |
| | NRRNSVVPAS WGETSASSVP GTCAATSAIG TYSSVTVTSW PSIVATGGTT |
| | 510 520 530 540 550 |
| | TTATPTGSGS VTSTSKTTAT ASKTSTSTSS TSCTTPTAVA VTFDLTATTT |
| | 560 570 580 590 600 |
| | YGENIYLVGS ISQLGDWETS DGIALSADKY TSSDPLWYVT VTLPAGESFE |
| | 610 620 630 640 |
| | YKFIRIESDD SVEWESDPNR EYTVPQACGT STATVTDTWR |
| Predicted Molecular Weight: | 68308.80 Daltons (irrespective of post-translational |
| | modifications (e.g., signal peptide cleavage, glycosylation, |
| | etc.) and lower molecular weight variants and products) |

| SEQ ID NO: | 7 |
|---|---|
| Trade Name: | Protease AM (BIO-CAT, Inc.) |
| Source organism: | Aspergillus melleus |
| Protein: | Basic amino-acid permease |
| Gene: | alp1 1 |
| Synonyms: | Alkaline protease 1 |
| IUBMB Enzyme | 3.4.21.63 |
| Commission (EC) No.: | |
| Length: | ≤404 amino acids |
| Amino Acid Sequence: | 10 20 30 40 50 |
| | MPSFKQTLLL LGAMLPAVFG APVENTRRAS EKIAGKYIVT FKSGIDTAKI |
| | 60 70 80 90 100 |
| | EQHTTWATNI HKRNLARRDA SDDSDLPVGI EKNFKIKNFA AYFGSFDDST |
| | 110 120 130 140 150 |
| | IEEIRKSADV AHIEEDQVWY LDALTTQTGA TWGLGSISHK GESSTSYVYD |
| | 160 170 180 190 200 |
| | SSAGEGTYGY VVDTGINVDH SEFGGRASLA YNAVGGQHVD SVGHGTHVAG |
| | 210 220 230 240 250 |
| | TIGGKTYGVS KKANLLSVKV FQGESSSTSI ILDGYNWAAN DIVSKSRTGK |
| | 260 270 280 290 300 |
| | AAINLSLGGG YSYAFNQAVE NAFDEGVLTV VAAGNENSDA GDTSPASAPN |
| | 310 320 330 340 350 |
| | ALTVAASTNR NARASFSNYG SVVDVFAPGQ DIKSAWIGSS SATNTISGTS |
| | 360 370 380 390 400 |
| | MATPHIVGLA IYLQALEGLT SPAAVTKRIK ELATSGVVTD VKGSPNLLAY |
| | NGAA |
| Predicted Molecular Weight: | 42230.81 Daltons (irrespective of post-translational |
| | modifications (e.g., signal peptide cleavage, glycosylation, |
| | etc.) and lower molecular weight variants and products) |

1. An enzyme mixture comprising a plurality of fungal enzymes from members of the genus Aspergillus and Candida, wherein the plurality of fungal enzymes comprises: (a) a protease obtained from A. oryzae, (b) a protease obtained from A. niger, (c) a protease obtained from A. melleus, (d) a lipase obtained from C. cylindracea or a lipase obtained from R. oryzae, (e) an amylase obtained from A. oryzae, and (f) a glucoamylase obtained from A. niger, wherein the enzyme mixture has exopeptidase, endopeptidase, lipase, alpha-amylase, and glucoamylase activities.

2. The enzyme mixture of claim 1, wherein the enzymes are present as follows:

| Enzyme | Units/Gram | |
|---|---|---|
| protease from A. oryzae | 10,000-1,000,000 | HUT/g |
| protease obtained from A. niger | 50-100,000 | SAPU/g |
| protease obtained from A. melleus | 10-10,000 | LAPU/g |
| lipase obtained from C. cylindracea or | 100-100,000 | FIP/g |
| lipase obtained from R. oryzae | | |
| amylase obtained from A. oryzae | 500-500,000 | SKB/g |
| glucoamylase obtained from A. niger | 5-5,000 | AGU/g |

3. The enzyme mixture of claim 1, wherein the enzymes are present as follows:

| Enzyme | Units/Gram | |
|---|---|---|
| protease from A. oryzae | 200,000-280,000 | HUT/g |
| protease obtained from A. niger | 1,000-1,400 | SAPU/g |
| protease obtained from A. melleus | 100-300 | LAPU/g |
| lipase obtained from C. cylindracea or | 10,000-14,000 | FIP/g |
| fungal lipase obtained from R. oryzae | | |
| amylase obtained from A. oryzae | 30,000-50,000 | SKB/g |
| glucoamylase obtained from A. niger | 75-125 | AGU/g |

4. The enzyme mixture of claim 1, wherein the enzymes are present as follows:

| Enzyme | Units/Gram | |
|---|---|---|
| protease from A. oryzae | 240,000 | HUT/g |
| protease obtained from A. niger | 1,200 | SAPU/g |
| protease obtained from A. melleus | 200 | LAPU/g |
| lipase obtained from C. cylindracea or | 12,000 | FIP/g |
| lipase obtained from R. oryzae | | |
| amylase obtained from A. oryzae | 40,000 | SKB/g |
| glucoamylase obtained from A. niger | 100 | AGU/g |

5. The enzyme mixture of any one of claims 1-4, wherein the ratio of the 3 fungal proteases: lipase:amylase:glucoamylase is:
a) 3600:120:400:1; or
b) 1800-5400:30-180:200-600:0.5-1.5;
as measured in hemoglobin unit tyrosine base units (HUT): Fédération Internationale Pharmaceutique units (FIP): Sandstedt, Kneen, and Blish method units (SKB): Amyloglucosidase units (AGU).

6. The enzyme mixture of any one of claims 1-5, wherein the enzyme mixture comprises at least four, five, six, or seven of SEQ ID NOs: 1, 2, 3, 4, 5, 6, and 7.

7. The enzyme mixture of any one of claims 1-5, wherein the enzyme mixture comprises SEQ ID NOs: 1, 2, 3, 4, 5, 6, and 7.

8. The enzyme mixture of any one of claims 1-5, wherein the enzyme mixture comprises SEQ ID NOs: 1, 2, 3, 4, 5, and 6.

9. The enzyme mixture of any one of claims 1-5, wherein the enzyme mixture comprises SEQ ID NOs: 1, 2, and 4-7.

10. The enzyme mixture of any one of claims 1-5, wherein the enzyme mixture comprises amino acid sequences which share at least 90, 91, 92, 93, 94, 95, 96, 97, 98 or 99% full-length sequence identity with any one of SEQ ID NOs: 1-7.

11. The enzyme mixture of any one of claims 1-10, in a dehydrated, powdered, granular or freeze dried form.

12. A dietary supplement comprising the enzyme mixture of any one of claims 1-11.

13. A dietary supplement comprising the enzyme mixture of any one of claims 1-11 and at least one additional enzyme comprising:
a) a lactase;
b) an alpha-galactosidase;
c) a beta-fructofuranosidase;
d) a cellulase;
e) papain; and/or
f) bromelain.

14. The dietary supplement of claim 12 or 13, wherein the dietary supplement is:
a) formulated as a tablet, capsule, chew, gummy, gum, or powder; and/or
b) a protein supplement or a nutritional supplement.

15. A method for increasing protein digestion, fat digestion, carbohydrate digestion, absorption of amino acids, absorption of fatty acids, absorption of glucose, and/or absorption of iron comprising administering the enzyme mixture of any one of claims 1-11, or the dietary supplement of any one of claims 12-14, to a subject at least once per day.

16. A method for decreasing the maximum plasma concentration of glucose comprising administering the enzyme mixture of any one of claims 1-11, or the dietary supplement of any one of claims 12-14, to a subject at least once per day.

17. A method for lessening the frequency, duration, and/or severity of gastrointestinal symptoms including burping, cramping, distension, flatulence, nausea, reflux, borborygmus, and/or diarrhea comprising: administering the enzyme mixture of any one of claims 1-11, or the dietary supplement of any one of claims 12-14, to a subject at least once per day.

18. A method for improving bowel function by improving stool frequency, stool consistency, and/or ease of stool passage comprising: administering the enzyme mixture of any one of claims 1-11, or the dietary supplement of any one of claims 12-14, to a subject at least once per day.

19. A method for improving sleep quality comprising: administering the enzyme mixture of any one of claims 1-11, or the dietary supplement of any one of claims 12-14, to a subject at least once per day.

20. The method any one of claims 15-19, wherein the dietary supplement is administered at least twice per day, at least three times per day, or at least four times per day.

21. The method of any one of claims 15-20, wherein the dietary supplement is administered to the subject before, after, or concurrently with a meal, food or beverage.

22. The method of any one of claims 15-21, wherein the dietary supplement is administered in an amount effective to increase the rate of absorption of essential amino acids, branched chain amino acids, fatty acids, glucose, and/or iron in the subject.

23. The method of any one of claims 15-22, wherein the dietary supplement is administered to the subject in an amount effective to improve muscle health.

24. The method of any one of claims 15-22, wherein the dietary supplement is administered to the subject in an amount effective to improve digestive health.

25. The method of any one of claims 15-24, wherein the subject is at least 40 years old.

26. The method of any one of claims 15-24, wherein the subject is at least 60 years old.

27. The method of any one of claims 15-26, wherein the subject consumes less than the daily recommended intake of nutrients, the subject experiences moderate gastrointestinal symptoms as measured by an average daily gastrointestinal score≥1 from the BF-GITFQ or average weekly daily gastrointestinal score≥1 from the GITQ, the subject has an average daily stool form score of 1, 2, 6, or 7 based on the Bristol Stool Form Scale, and/or the subject experiences poor sleep quality.

28. The method of any one of claims 15-27, wherein the enzymes are present as follows:

| Enzyme | Units/Dose | |
|---|---|---|
| protease from A. oryzae | 20,000-100,000 | HUT/dose |
| protease obtained from A. niger | 50-400 | SAPU/dose |
| protease obtained from A. melleus | 15-100 | LAPU/dose |
| lipase obtained from C. cylindracea or | 1,000-5,000 | FIP/dose |
| lipase obtained from R. oryzae | | |
| amylase obtained from A. oryzae | 5,000-20,000 | SKB/dose |
| glucoamylase obtained from A. niger | 10-100 | AGU/dose |

29. The method of any one of claims 15-27, wherein the enzymes are present as follows:

| Enzyme | Units/Dose | |
|---|---|---|
| protease from A. oryzae | 60,000 | HUT/dose |
| protease obtained from A. niger | 300 | SAPU/dose |
| protease obtained from A. melleus | 50 | LAPU/dose |
| lipase obtained from C. cylindracea or | 3,000 | FIP/dose |
| lipase obtained from R. oryzae | | |
| amylase obtained from A. oryzae | 10,000 | SKB/dose |
| fungal glucoamylase obtained from A. niger | 25 | AGU/dose |
